(19) United States
(12) Patent Application Publication
(10) Pub. No.: US 2020/0062855 A1
(43) Pub. Date: Feb. 27, 2020

(54) COMPOSITIONS AND METHODS OF PROMOTING WOUND HEALING

(71) Applicant: CASE WESTERN RESERVE UNIVERSITY, Cleveland, OH (US)

(72) Inventors: M. Edward Medof, Cleveland, OH (US); Timothy Kern, Cleveland, OH (US); Lorna Kang, Cleveland, OH (US); Maryo Kohen, Cleveland, OH (US); Faruk Orge, Cleveland, OH (US)

(21) Appl. No.: 16/660,473

(22) Filed: Oct. 22, 2019

Related U.S. Application Data
(63) Continuation-in-part of application No. 13/350,402, filed on Jan. 13, 2012.
(60) Provisional application No. 62/749,065, filed on Oct. 22, 2018.

(51) Int. Cl. C07K 16/28; A61P 17/02

(52) U.S. Cl. CPC C07K 16/2896; C07K 2317/76; A61P 17/02

(57) ABSTRACT

A method of promoting wound healing and/or treating wounds in a subject in need thereof includes administering to cells proximate or about the periphery of the wound at least one agent that enhances C3aR and/or C5aR signaling of the cells.

### RELATED APPLICATION

This application claims priority from U.S. Provisional Application No. 62/749,065, filed Oct. 22, 2018, this application is also a Continuation-in-Part of Ser. No. 13/350,402, filed Jan. 13, 2012, the subject matter, which is incorporated herein by reference in its entirety.

### BACKGROUND

Multiple host processes have been implicated in wound repair. Important among them are growth factor signaling, upregulation of integrins, and the production of pro-mitotic cytokines. Examples of each include the production of vascular endothelial cell growth factor-A (VEGF-A) and/or fibroblast growth factor (FGF) which induce growth signaling through their respective receptor tyrosine kinases (RTKs), the upregulation of integrins-α1 and -α2 which provide cell-cell adhesion needed for tissue repair, and the production of IL-6 and consequent IL-6 receptor (IL-6R) transduction, which triggers Janus kinase-(JAK) activation and nuclear translocation of the signal transducer and activation of transcription 3 (STAT3) that participates both in growth relevant cell signaling and gene transcription.

A common signaling node downstream of all of these processes is assembly of inner leaflet phosphatidylinositol 3,4,5 trisphosphate [PtdIns (3,4,5)P₃]. Virtually all steps connected with its downstream signaling and regulation have been linked to healing. These include downregulation of the phosphatase and tensin homolog (PTEN) which disassembles it and upregulation of phosphatidyl inositol-3 kinase-α (PI-3Kα) which assembles it. Downstream of the augmented PtdIns (3,4,5)P₃ generation, they include the phosphorylation and nuclear translocation of NF-κB and STAT3, which induce the transcription of pro-mitotic genes, the activation of extracellular signal-regulated kinase (ERK), and the phosphorylation of protein kinase B (AKT) and its downstream signaling to the mammalian target of Rapamycin (mTOR), which triggers cell cycle entry that initiates cell division. Because of the multiplicity of these linkages as well as other growth inductive processes with healing, a cellular process jointly interconnecting their induction and function has not been elucidated. Identification of such a process, if it exists, could open a way to more rapidly restore function following injury as well as reduce wound complications.

The complement system for many years was widely regarded as existing solely in plasma, deriving exclusively from the liver, and functioning only in innate immunity. In prior studies of immune cell activation, we found that interacting antigen presenting dendritic cells (DCs) and cognate CD4⁺ cells locally generate C3a and C5a from endogenously synthesized complement. The locally produced C3a and C5a ligate C3a and C5a receptors (C3ar1/C5ar1) on each partner and the resulting autocrine C3ar1/C5ar1 signaling drives T cell proliferation. The intensity of C3a/C5a production and consequent intensity of C3ar1/C5ar1 signaling is governed by the cell associated complement regulator decay accelerating factor (DAF or CD55). When restraint by DAF is lifted, C3a/C5a production is increased and T cell proliferation is accelerated, whereas when DAF expression is heightened, C3a/C5a production and T cell proliferation are repressed. Studies with mice deficient in DAF (Daf1^{-/-} mice) or devoid of the C3/C5 sources (C3^{-/-}C5^{-/-} mice) of C3a/C5a or their C3ar1/C5ar1 receptors (C3ar1^{-/-}C5ar1^{-/-} mice) validated this growth relevant relationship.

### SUMMARY

Embodiments described herein relate to a method of promoting wound healing and/or treating a wound in a subject in need thereof by administering to cells proximate or about the periphery of a wound of the subject at least one agent that enhances C3aR and/or C5aR signaling of the cells. It was found that disrupting decay-accelerating factor (DAF) (CD55) function of cells proximate and/or about the periphery of a wound of a subject uniformly accelerated healing of the wound, whereas disabling C3ar1/C5ar1 of the cells had the opposite effect. The mechanism underlying these findings is that autocrine C3ar1/C5ar1 signaling operates in non-immune cells as in immune cells, and that the intensity of this GPCR signaling is controlled by DAF. In the absence of DAF's restraint on local C3a/C5a generation, potentiated C3ar1/C5ar1 signaling promotes cellular growth, viability, and/or mitosis. Conversely, abrogated C3ar1/C5ar1 signaling represses mitotic activity.

In some embodiments, the agent substantially increases, enhances, potentiates and/or promotes the interaction of at least one of C3a or C5a with the C3a receptor (C3aR) and C5a receptor (C5aR). The agent can include at least one of DAF antagonist, C3aR agonist and/or a C5aR agonist.

In some embodiments, the DAF antagonist can include an anti-DAF antibody or antigen binding fragment thereof that binds to DAF to reduce DAF inhibition and/or degradation of C3 convertase and/or C5 convertase. In one example, the anti-DAF antibody or antigen binding fragment thereof can bind to CCP2 and/or CCP3 of DAF.

In other embodiments, the agent can include DAF interfering RNA that inhibits expression of DAF.

In still other embodiments, the agent can include a C3aR agonist and/or a C5aR agonist that enhances C3aR and/or C5aR signaling of the cells.

In some embodiments, the agent can be administered to the wound of the subject at an amount effective to promote at least one of growth, viability, or mitosis of cells proximate or about the periphery of the wound.

In some embodiments, the wounds include acute injuries or wounds, damage to bodily tissues, injuries sustained during medical procedures, trauma-induced injuries, ulcers, post-surgical injuries, chronic wounds, acne, dermatitis, wounds following dental surgery, periodontal disease, and tumor associated wounds.

In other embodiments, the wounds can include at least one of thermal burns, chemical burns, radiation burns, burns caused by excess exposure to ultraviolet radiation, damage to bodily tissues as a result of labor and childbirth, cuts, incisions, excoriations, injuries sustained as result of accidents, pressure ulcers, diabetic ulcers, plaster ulcers, decubitus ulcer, post-surgical injuries, pressure sores, bedsores, acne, impetigo, intertrigo, folliculitis, or eczema.

### BRIEF DESCRIPTION OF THE DRAWINGS

The foregoing and other features of the invention will become more apparent upon a consideration of the following description taken in connection with the accompanying drawings wherein:

FIG. 1 is a schematic diagram illustrating growth factor and amplification through complement receptor signaling.

FIG. 2 is a schematic diagram illustrating that when C3aR/C5aR signal transduction is antagonized either pharmacologically, immunologically, or genetically, cell growth and progression from G0 to G2 is blocked.

FIG. 3 illustrates a chart showing the effect of C3aR/C5aR antagonism on 7 RTKs and β1-adreneric receptor in respective cell types.

FIG. 4 illustrates a chart showing autocrine C3aR/C5aR signaling is essential for EC viability. bEnd.3 and MS-1 cells were incubated for 24 hours in EC growth medium, and locally produced C3a and C5a in culture supernatants quantitated by ELISA.

FIG. 5 illustrates a chart showing Bcl-2, Bclx-L, Bax, and Bim mRNA expression levels in bEnd.3 cells incubated for 8 hr with C3aR-A/C5aR-A (10 ng/ml each) or anti-C3a/C5a (1 ug/ml each).

FIG. 6 illustrates a chart showing Annexin V positivity of bEnd.3 cells and MS-1 cells administered C3aR-A/C5aR-A (10 ng/ml each).

FIG. 7 illustrates a chart showing complement mRNA transcription (Left, data shown for C3 mRNA) in HUVEC cells following C3a or C5a administration or hypoxia. Hypoxia was induced by FCCP and IAA in HUVEC for 1 hr and C3 mRNA levels were quantified by qPCR (Right).

FIG. 8 illustrates plots showing growth of bEnd.3 cells following administration of C3aR-A/C5aR-A signaling and/or VEGF.

FIG. 9 illustrates plots showing growth of MS-1 cells following administration of C3aR-A/C5aR-A signaling and/or VEGF.

FIG. 10 illustrates plots showing growth of HUVECs incubated with C3aR-A/C5aR-A (10 ng/ml each) or anti-C3/anti-C5 mAbs (1 μg/ml each) at 24, 28 and 72 hr assayed.

FIG. 11 illustrates plots showing expression of C3 and C5 mRNA of bEnd.3 cells incubated for 1 hr with VEGF-A (30 ng/ml).

FIG. 12 illustrates a chart showing C3a/C5a production of bEnd.3 cell or MS-1 cells treated with VEGF and C3aR-A/C5aR-A.

FIG. 13 illustrates a chart showing Annexin V positivity in primary ECs added of C3aR-A/C5aR-A (10 ng/ml each).

FIG. 14 illustrates a chart showing C3, fB, fD, C5, C3aR, and C5aR levels of primary ECs were incubated for 30 min with VEGF-A (30 ng/ml).

FIG. 15 illustrates images showing primary ECs isolated from aortic rings of Daf1^{-/-}, WT and C3aR^{-/-}C5aR^{-/-} mice grown in EC growth medium for 2 wk and photographed following the first passage.

FIG. 16 illustrates a plot showing cell numbers of 5×10⁵ ECs of each genotype cultured in EC growth medium after 24, 48, and 72 hr.

FIG. 17 illustrates a chart showing mRNA expression of C3, fB, fD, and C5 by cells from whole aorta of each identified genotype.

FIG. 18 illustrates plots showing growth of WT and C3aR^{-/-}C5aR^{-/-} incubated with VEGF-A (30 ng/ml) at 24, 48, and 72 hr assayed.

FIG. 19 illustrates images showing autocrine C3aR/C5aR signaling in ECs is essential for HUVEC tube formation and corneal neovascularization. HUVEC were plated with EBM-2 Basal Medium without supplemental growth factors, with VEGF-A, or with VEGF-A plus C3aR-A/C5aR-A.

FIG. 20 illustrates images showing male mice were injected subcutaneously with 1×10⁶ RM1 prostate cancer cells. Tumors were collected 10 days after injection and were weighed (Top, n=8). Representative images of immunostaining for CD31 in tumor sections of WT, Daf1^{-/-}, and C3aR^{-/-}C5aR^{-/-} mice, showing CD31 expression in new vessels (Middle). CD31-positive areas were quantified in 5-10 independent fields per tumor implant (Bottom).

FIG. 21 illustrates an immunoblot of VEGFR2 phosphorylation. C5a or VEGF-A was added to serum starved primary cultures of WT murine ECs in the absence or presence of C3aR-A/C5aR-A and VEGFR2 phosphorylation assessed by immunoblotting with anti p-T1094/T1095 mAb.

FIG. 22 illustrates a chart showing quantitation of bands in FIG. 21.

FIG. 23 illustrates a plot showing the expression of VEGF from the second passage of primary ECs from WT and C3aR^{-/-}C5aR−/−.

FIG. 24 illustrates the expression of VEGFR2 of primary ECs starved in DMEM/F12 with 0.5% FBS for 24 hrs. The supernatants and cell lysates were collected and analyzed for VEGF content by ELISA. The results were normalized by protein concentration.

FIG. 25 illustrates plots showing growth of serum starve primary cultures of WT murine ECs in the absence or presence of SU5416 or C5a. The cell proliferation was quantified by Trypan blue exclusion.

FIG. 26 illustrates a chart showing the expression of Integrinβ3 from the passage 3-4 of primary EC of WT and C3aR^{-/-}C5aR^{-/-} measured by FACS.

FIG. 27 illustrates plots showing the growth of ECs in response to VEGF induces C3aR/C5aR signaling via an IL-6 and Stat3 dependent mechanism. VEGF-A was added to serum starved MS-1 cells in the absence or presence of anti-IL-6 neutralizing mAb (2 μg/ml) and growth was quantified at 24, 28 and 72 hr.

FIG. 28 illustrates plots showing the growth of serum starved MS-1 cells in the absence or presence of C3aR-A/C5aR-A (10 ng/ml each) and IL-6 (10 ng/ml) at 24, 48 and 72 hr.

FIG. 29 illustrates a chart showing phosphor-Stat3 of serum starved MS-1 cells incubated for 10 min at 37° C. with VEGF-A or IL-6 in the absence or presence of anti-IL-6 mAb or C3aR-A/C5aR-A.

FIGS. 30(A-B) illustrate plots showing (A) NIH-3T3 cells were in cubated as indicated cell counted over 72 hrs. (B) CTLL IL-2 dependent cells were incubated as indicated and counted over 72 hrs.

FIGS. 31(A-B) illustrate charts showing: (A) C5a (17 ng/mL) added to NIH-3T3 cells. Bars represent 72 hr counts. (B) PDGF-AA was added to NIH-3T3 cells and a 72 hr culture supernatants were assayed for C5a by ELISA.

FIG. 32 illustrates plots showing SMCs from different knockouts were incubated with PDGF-AA and cell numbers determined each day.

FIGS. 33(A-D) illustrate charts showing qRT-PCR analysis of C3, factor B and factor D transcripts from HUVEC under hypoxia. A) HUVEC treated with TNF-α, IL-1, and IFN-γ. Blue bars represent samples after treatment. B-C) HUVEC stimulated with C3a (10 ng/ml for 2 hr) or C5a (10 ng/ml for 30 min.) Blue bars represent control without C3a/C5a stimulation while red bars represent samples after stimulation. D) HUVEC incubated for 1 hr with FCCP+IAA. Blue bars represent control without hypoxia treatment while yellow bars represent two samples after hypoxia treatment.

FIG. 34 illustrates a chart showing HUVECs stimulated with simvastin, and assayed for DAF and KLF4 mRNA.

FIGS. 35(A-D) illustrate images and charts showing: A) Verhoeffelastin stain 14 day after femoral artery wire injury (original magnification 10×). B) Intima area:media area ratio 14 d after injury. C) Medical Leukocyte (% CD45-positive cells) accumulation 14 d after injury. D) Cellular proliferation (% BrdU-positive cells) in the media 14 day after injury.

FIG. 36(A-C) illustrate a chart showing recovery from ear puncture and autologous skin transplant. (A) Equal size ear lobe punctures were made in Daf1^{-/-}, WT and C3ar1^{-/-}C5ar1^{-/-} mice (6 each) with a 5 mm punch. Wound closure was measured over 29 d. Percentage decrease in size is shown on d 7, 21, and 29. (B) Following shaving and depilation of backs, a 2×2 cm of skin was removed. Immediately thereafter a transplant from the donor mouse (cut to the same size) was secured in place. Mice were euthanized 14 d later and frozen sections of the skin transplant were stained with rat anti mouse CD31 mAb followed by Alexa Fluor 594 labeled goat anti-rat CD31 (red). Anti-rat IgG2a was included as a control. Nuclei were stained with Hoechst (blue). Representative images are shown at 20× magnification. (C) Blood vessel areas in the transplants in panel B for the three genotypes were quantified by NIS-Elements. Total areas (red) in each image were measured.

FIGS. 37(A-B) illustrate recovery from burn wound and corneal denudation. (A) An insulated 8 mm diameter steel rod was heated to 120° C. A uniform full-thickness (8 mm) burn wound was made in Daf1^{-/-}, WT, and C3ar1^{-/-}C5ar1^{-/-} mice (6 each group) by placing the rod on the shaved area for 20 s. The decrease in the size of the burn wound area was compared over a 9 day period. (B) A uniform 0.5 cm deep (5-6 cell deep) circular 1.5 mm diameter layer of anesthetized corneal epithelium from Daf1^{-/-}, WT, and C3ar1^{-/-}C5ar1^{-/-} mice (6 each group) was removed with a 1.5 mm trephine employing an Algerbrush II with a 0.5 mm Burr. After recovery from anesthesia, corneas were stained with fluorescein to quantitate wound size. Wound healing was compared at 4 h, 12 h and 24 h.

FIGS. 38(A-B) illustrate images and charts showing the ability of DAF blockade to accelerate wound healing. (A) A uniform full-thickness burn wound was made in WT mice as in FIG. 2 panel A. Wounds were covered for 24 hr with bandages (3M) presoaked in mouse anti-mouse Daf1 CCP23 anti-plasma (6 mice) or presoaked with pre-immunization plasma (6 mice). Burn wound size was quantitated as in FIG. 37A. The experiment was repeated 3 times with consistent results (p<0.05). FIG. 37B. A uniform circular layer of corneal epithelium was removed from WT mice (6 mice) as in FIG. 37B. The mice were treated with mouse anti-mouse Daf1-CCPs23 antiserum or pre-immunization serum as control. After recovery from anesthesia, corneas were stained with fluorescein and wound healing measured at 12 h and 24 h. The experiment was repeated 3 times with consistent results (p<0.005).

FIG. 39 illustrates a chart showing the preparation of anti-mouse Daf1 CCPs23 mAbs. Titers of anti-DAF plasmas from different Daf1 CCP23^{-/-} mice immunized with full length recombinant mouse DAF protein. Hybidomas were prepared conventionally (fusing spleen cells from the immunized Daf1 CCPs23^{-/-} mice with myeloma cells).

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994. The definitions provided herein are to facilitate understanding of certain terms used frequently herein and are not meant to limit the scope of the application described herein.

As used herein, the term “polypeptide” refers to an oligopeptide, peptide, or protein sequence, or to a fragment, portion, or subunit of any of these, and to naturally occurring or synthetic molecules. The term “polypeptide” also includes amino acids joined to each other by peptide bonds or modified peptide bonds, i.e., peptide isosteres, and may contain any type of modified amino acids. The term “polypeptide” also includes peptides and polypeptide fragments, motifs and the like, glycosylated polypeptides, and all “mimetic” and “peptidomimetic” polypeptide forms.

As used herein, the term “polynucleotide” refers to oligonucleotides, nucleotides, or to a fragment of any of these, to DNA or RNA (e.g., mRNA, rRNA, tRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, siRNAs, microRNAs, and ribonucleoproteins. The term also encompasses nucleic acids, i.e., oligonucleotides, containing known analogues of natural nucleotides, as well as nucleic acid-like structures with synthetic backbones.

As used herein, the term “antibody” refers to whole antibodies, e.g., of any isotype (IgG, IgA, IgM, IgE, etc.), and includes fragments thereof which are also specifically reactive with a target polypeptide. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain polypeptide. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term “antibody” also includes polyclonal, monoclonal, or other purified preparations of antibodies, recombinant antibodies, monovalent antibodies, and multivalent antibodies. Antibodies may be humanized, and may further include engineered complexes that comprise antibody-derived binding sites, such as diabodies and triabodies.

As used herein, the term “complementary” refers to the capacity for precise pairing between two nucleobases of a polynucleotide and its corresponding target molecule. For example, if a nucleobase at a particular position of a polynucleotide is capable of hydrogen bonding with a nucleobase at a particular position of a target polynucleotide (the target nucleic acid being a DNA or RNA molecule, for example), then the position of hydrogen bonding between the polynucleotide and the target polynucleotide is considered to be complementary. A polynucleotide and a target polynucleotide are complementary to each other when a sufficient number of complementary positions in each molecule are occupied by nucleobases, which can hydrogen bond with each other. Thus, “specifically hybridizable” and “complementary” are terms which can be used to indicate a sufficient degree of precise pairing or complementarity over a sufficient number of nucleobases such that stable and specific binding occurs between a polynucleotide and a target polynucleotide.

As used herein, the term “subject” refers to any warm-blooded organism including, but not limited to, human beings, rats, mice, dogs, goats, sheep, horses, monkeys, apes, rabbits, cattle, etc.

As used herein, the terms “treatment,” “treating,” or “treat” refers to any specific method or procedure used for the cure of, inhibition of, prophylaxis of, reduction of, elimination of, or the amelioration of a disease or pathological condition including, for example, wounds, central nervous system injuries, peripheral nervous system injuries, and ischemia.

As used herein, the term “effective amount” refers to a dosage of an agent described herein administered alone or in conjunction with any additional therapeutic agents that are effective and/or sufficient to provide treatment of a disease or pathological condition, such as wounds, central nervous system injuries, peripheral nervous system injuries, and ischemia. The effective amount can vary depending on the subject, the disease being treated, and the treatment being affected.

As used herein, the term “therapeutically effective amount” refers to that amount of an agent described herein administered alone and/or in combination with additional therapeutic agents that results in amelioration of symptoms associated with a disease or pathological condition, such as wounds, central nervous system injuries, peripheral nervous system injuries, and ischemia.

As used herein, the terms “parenteral administration” and “administered parenterally” refers to modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

As used herein, the terms “pharmaceutically or pharmacologically acceptable” refer to molecular entities and compositions that do not produce an adverse, allergic or other untoward reaction when administered to an animal, or a human, as appropriate. Veterinary uses are equally included within the invention and “pharmaceutically acceptable” formulations include formulations for both clinical and/or veterinary use.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. For human administration, preparations should meet sterility, pyrogenicity, general safety and purity standards as required by FDA Office of Biologics standards. Supplementary active ingredients can also be incorporated into the compositions.

As used herein, “Unit dosage” formulations are those containing a dose or sub-dose of the administered ingredient adapted for a particular timed delivery. For example, exemplary “unit dosage” formulations are those containing a daily dose or unit or daily sub-dose or a weekly dose or unit or weekly sub-dose and the like.

Embodiments described herein relate to methods and compositions of promoting wound healing and/or treating wounds in a subject in need thereof. The methods can include administering to cells expressing C3a receptor (C3aR) and C5a receptor (C5aR) of the wound or about the periphery of the wound a therapeutically effective amount of at least one agent that increases, enhances, potentiates, and/or promotes C3aR and/or C5aR signaling as well as growth, viability, and/or mitosis of cells proximate or about the periphery of the wound.

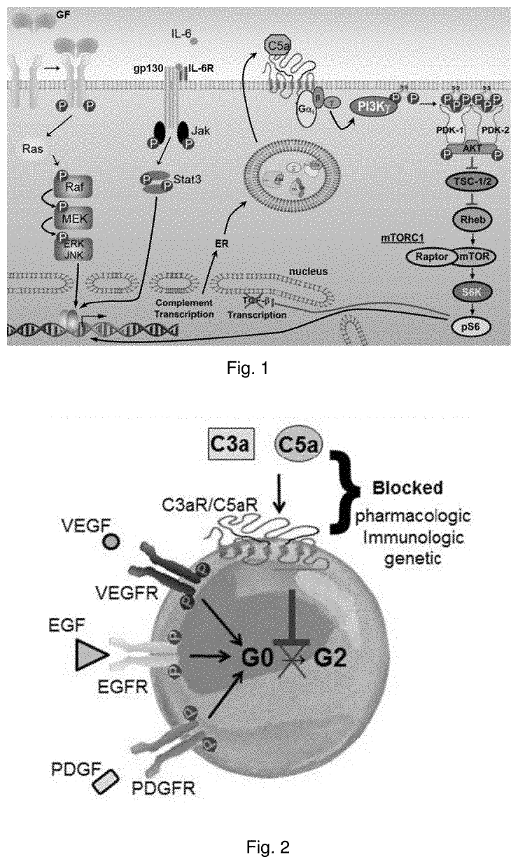

It was found that C3aR/C5aR signaling resulting from C3a/C5a endogenously produced by the same cell plays a central role in the function of many, if not most, receptor tyrosine kinases (RTKs) and some G protein coupled receptors, (GPCRs), affecting viability and cell proliferation, and tissue homeostasis and function (FIG. 1). Studies of T cell activation during interaction of antigen presenting dendritic cells (DCs) with cognate T cells showed that both partners locally synthesize complement and that paracrine/autocrine interactions of locally produced C3a/C5a with C3aR/C5aR on both partners provide costimulatory and survival signals to the T cells. The findings indicated that potentiated C3aR/C5aR signaling as contrasted to disabled C3aR/C5aR signaling in T cells (i.e., proliferation vs. PCD) is controlled by surface DAF.

We tested whether this autocrine signaling operates in other cell types and found that this signaling supports the viability of primary cultured ECs, SMCs, embyonic fibroblasts (pMEFs), breast and gastrointestinal epithelial cells (EPCs) and that its interruption in all cases induces apoptosis. We found that the mitotic and/or viability effects of seven RTKs and one GPCR depend on autocrine C3aR/C5aR signaling. We also found that IL-6 receptor (IL-6R) and Stat3 are involved in hormone and cytokine growth induction as well as local C5a in the mitotic effects of thrombin.

We also found that RTK signaling interconnects with autocrine C3aR/C5aR signaling and that blockade of either the receptors or their ligands completely abrogated EGF induced growth. Prompted by this result, we performed parallel studies of VEGF-A and PDGF-AA growth induction initially in murine EC lines (bEND.3 and MS-1) and in NIH-3T3 cells, respectively. These experiments surprising yielded near identical results.

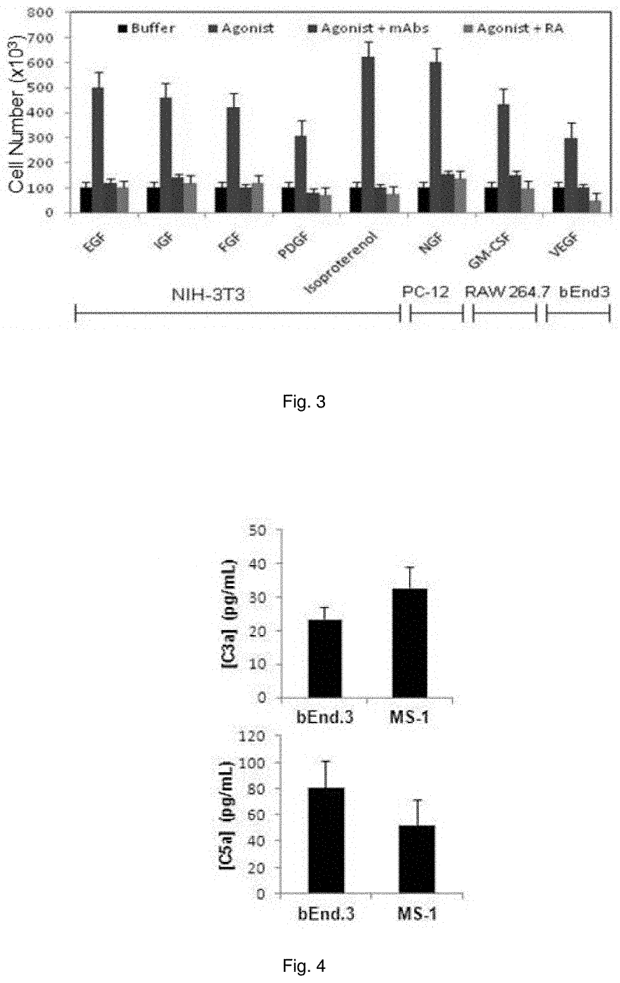

Because this dependence of growth factor responsiveness on C3aR/C5aR signaling could be indirect, i.e., a consequence of its requirement for viability, we performed cell cycle assays. Adding C5a to serum starved NIH-3T3 cells, bEND.3 ECs, or TC-1 cancer cells caused transition from G0 into G2 identically to that of adding EGF to NIH-3T3 or TC-1 cells or adding VEGF-A to bEND.3 ECs (FIGS. 2 and 3). Importantly, including C3aR-A/C5aR-A together with EGF or with VEGF-A markedly blunted or abolished triggering of the cell cycle by EGF and VEGF-A, suggesting that autocrine C3aR/C5aR signals not only limit apoptosis but are needed for cell cycle progression.

To gain mechanistic insight, we focused on VEGF-A growth induction through VEGFR2 in ECs. As a first test of whether VEGFR2 growth induction is interconnected with upregulated C3aR/C5aR signaling, we examined the effect of added VEGF-A on local complement production by the MS-1 and bEND.3 EC cell lines. ELISAs of their culture supernatants showed that VEGF-A increased local C3a as well as C5a production, both ˜8-fold and that both increases were abolished by the inclusion of C3aR-A/C5aR-A. Adding C5a to serum starved HUVEC caused transition from G0 into G2 identically to adding VEGF-A, whereas C3aR/C5aR blockade prevented VEGF-A triggering of the cell cycle. These findings together with the dependence VEGF-A growth induction on C3aR/C5aR signaling indicated that VEGFR2 signals amplify C3aR/C5aR signal transduction and that amplification of this autocrine GPCR signaling integrates with VEGFR2 growth signals. Consistent with the increased local C3a/C5a production, VEGF-A upregulated mRNA transcripts of all of the components/receptors associated with autocrine C3aR/C5aR signaling in primary cultured aortic ECs, whereas antagonizing C3aR/C5aR abrogated the up-regulations and induced markers of apoptosis.

To determine if the linkage between VEGF-A and C3aR/C5aR signaling in ECs involves IL-6, we incubated MS-1 ECs with 1) VEGF-A alone, IL-6 alone, or VEGF-A plus anti-IL-6 mAb, or with 2) IL-6 alone or IL-6 plus C3aR-A/C5aR-A, and assayed cell growth. IL-6 induced EC growth comparably to VEGF-A and VEGF-A's growth induction was abolished by anti-IL-6 mAb. The EC induced growth by IL-6, like that of VEGF-A, was abolished by C3aR-A/C5aR-A. Both VEGF-A and IL-6 induced Stat3 phosphorylation. Importantly, the Stat3 phosphorylation in both cases was abolished by C3aR/C5aR antagonism. Relevant to this, VEGF-A treatment or WT aortic ECs upregulated C3/C5 and increased local C3a/C5a generation as found for MS-1 ECs, but neither change occurred in the presence of anti-IL-6 mAb or the JAK1 inhibitor. These findings thus indicate that VEGFR2 signaling interconnects with C3aR/C5aR signaling via a process involving induction of IL-6 and activation of Stat3.

We surprisingly found that disrupting decay-accelerating factor (DAF) (CD55) function of cells proximate and/or about the periphery of a wound of a subject uniformly accelerated healing of the wound, whereas disabling C3ar1/C5ar1 of the cells had the opposite effect. The mechanism underlying these findings is that autocrine C3ar1/C5ar1 signaling operates in non-immune cells as in immune cells, and that the intensity of this GPCR signaling is controlled by DAF. In the absence of DAF's restraint on local C3a/C5a generation, potentiated C3ar1/C5ar1 signaling promotes cellular growth, viability, and/or mitosis. Conversely, abrogated C3ar1/C5ar1 signaling represses mitotic activity. It was further found that adding anti-DAF antibodies to the wound area in burns and to injured corneas accelerated healing, potentially having therapeutic relevance to many types of tissue injuries.

Accordingly, based at least in part on these findings, in some embodiments a population of cells expressing C3a receptor (C3aR) and C5a receptor (C5aR) proximate and/or about the periphery of a wound can be contacted (e.g., directly or locally) with a therapeutically effective amount of an agent that promotes C3aR and/or C5aR signaling of the cells and, optionally, promotes response of the cells to a growth factor. This promotion of C3aR/C5aR signaling can enhance viability, function, or mitosis of the cells proximate and/or about the periphery of the wound and promote wound healing and/or treat the wound.

In some embodiments, an increase in growth, viability, and/or mitosis of a cell expressing C3a receptor (C3aR) and C5a receptor (C5aR) and optionally at least one growth factor receptor (e.g., RTK), can be increased, promoted, or enhanced by administering to the cell an agent that increases, promotes, potentiates, and/or enhances the activity of a complement component and, in turn, C3aR and/or C5aR signaling of the cells. By increasing the activity of a complement component, it is meant that the activity of the complement component may be enhanced. For example, an increase in the functioning of a C3/C5 convertase may promote cleavage of C5 and C3 into C5a and C3a, respectively. An increase in the functioning of C5, C3, C5a and/or C3a polypeptides may increase or promote the ability of C5a and C3a to bind C5aR and C3aR, respectively. An increase in Factor B, Factor D, properdin, Bb, Ba and/or any other protein of the complement pathway that is used in the formation of C3 convertase, C5 convertase, C5, C3, C5a and/or C3a may increase the ability of C5a and C3a to be formed and bind to C5aR and C3aR, respectively. Additionally, an inhibition or reduction in the functioning of a DAF may similarly reduce or eliminate the DAF mediated degradation of C3 convertase and/or C5 convertase and enhance the formation C5a and C3a and binding C5aR and C3aR, respectively.

In some embodiments, an agent that promotes or stimulates C3aR and/or C5aR signaling of the cells can include an antibody or antigen binding fragment thereof directed against a complement component that can decrease or inhibit the formation of C3a and/or C5a (e.g., anti-DAF or) and/or decrease or inhibit C5a/C3a-05aR/C3aR interactions. In one example, the antibody or antigen binding fragment can be directed against or specifically bind to an epitope, an antigenic epitope, or an immunogenic epitope of DAF. The term “epitope” as used herein can refer to portions of DAF having antigenic or immunogenic activity. An “immunogenic epitope” as used herein can include a portion of DAF that elicits an immune response in a subject, as determined by any method known in the art. The term “antigenic epitope” as used herein can include a portion of a polypeptide to which an antibody can immunospecifically bind as determined by any method well known in the art.

Examples of antibodies directed against DAF are known in the art. For example, mouse monoclonal antibodies directed against DAF can include those that bind to CCP2 and/or CCP3 of DAF that are described in the example. Other examples of antibodies directed against DAF (or antibodies which specifically bind to DAF) include the murine monoclonal antibodies IA10, IIH6 and VIIIA7 as described in WO86/07062 published Dec. 4, 1986 and expressly incorporated herein by reference; the human antibodies designated LU30, LU13 and LU20 as described in U.S. Patent Application Publication No. 2003/0219434; the murine 110 and BRIC 216 monoclonal antibodies directed against DAF as described in WO99/43800; the murine 791T36 antibody directed against the 791Tgp72 antigen (ATCC HB9173; WO99/43800); the D17 murine antibody described in Hara et al. Immunol. Lett. 37:145-152 (1993) which binds DAF on blood cells; the human SC-1 antibody (Vollmers et al. Cancer 76(4): 550-558 (1995); Vollmers et al. Cancer Research 49: 2471-2476 (1989); Vollmers et al. Oncology Reports 5:549-552 (1998); and Hensel et al. Cancer Research 59:5299-5306 (1999)), as well as variants of any one of the above antibodies. Antibody variants including amino acid sequence variants (e.g., affinity matured antibodies and humanized variants of murine antibodies), glycosylation variants with altered effector function, etc.

In other embodiments, the agent that promotes or stimulates C3aR and/or C5aR signaling of the cells can include RNA interference (RNAi) polynucleotides that induce knockdown of an mRNA encoding DAF. For example, an RNAi polynucleotide can comprise a siRNA capable of inducing knockdown of an mRNA encoding DAF.

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules or other RNA molecules including a double stranded feature and able to be processed to siRNA in cells, such as hairpin RNA moieties.

The term “loss-of-function,” as it refers to genes inhibited by the subject RNAi method, refers to a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response.

As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to replicable nucleic acid constructs used to express (transcribe) RNA which produces siRNA moieties in the cell in which the construct is expressed. Such vectors include a transcriptional unit comprising an assembly of (I) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The choice of promoter and other regulatory elements generally varies according to the intended host cell. In general, expression vectors of utility in recombinant DNA techniques are often in the form of “plasmids” which refer to circular double stranded DNA loops, which, in their vector form are not bound to the chromosome. As described herein, the terms “plasmid” and “vector” are used interchangeably as the plasmid is the most commonly used form of vector. However, this disclosure is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are siRNAs. These nucleic acids are around 19-30 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex.

The siRNA molecules can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

Examples of a siRNA molecule directed to an mRNA encoding a DAF are known in the art. For instance, human DAF siRNA can have the nucleic acid sequences of 5′ gaagaguucugcaaucgua 3′ (sense) (SEQ ID NO: 1) and 5′ uacgauugcagaacucuuc 3′ (antisense) (SEQ ID NO: 2).

In other embodiments, the RNAi construct can be in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects, which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Such hairpin RNAs are engineered in cells or in an animal to ensure continuous and stable suppression of a desired gene. It is known in the art that siRNAs can be produced by processing a hairpin RNA in the cell.

In yet other embodiments, a plasmid can be used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an example of a vector for bi-directional transcription of a transgene to yield both sense and antisense RNA transcripts of the same transgene in a eukaryotic cell. Accordingly, in certain embodiments, the a recombinant vector can have the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

RNAi constructs can comprise either long stretches of double stranded RNA identical or substantially identical to the target nucleic acid sequence or short stretches of double stranded RNA identical to substantially identical to only a region of the target nucleic acid sequence. Examples of methods of making and delivering either long or short RNAi constructs can be found, for example, in WO01/68836 and WO01/75164.

Examples RNAi constructs that specifically recognize a particular gene or a particular family of genes, can be selected using methodology outlined in detail above with respect to the selection of antisense oligonucleotide. Similarly, methods of delivery RNAi constructs include the methods for delivery antisense oligonucleotides outlined in detail above.

In some embodiments, a lentiviral vector can be used for the long-term expression of a siRNA, such as a short-hairpin RNA (shRNA), to knockdown expression of DAF in cells proximate and/or about the periphery of a wound. Although there have been some safety concerns about the use of lentiviral vectors for gene therapy, self-inactivating lentiviral vectors are considered good candidates for gene therapy as they readily transfect mammalian cells.

Moreover, it will be appreciated that other antibodies, small molecules, and/or peptides that reduce or inhibit the formation of DAF and/or that increase, enhance, or promote interactions C5a and/or C3a with C5aR and C3aR on the cells expressing C3a receptor (C3aR) and C5a receptor (C5aR) and optionally at least one growth factor receptor (e.g., RTK) can be used as an agent in accordance with the method described herein. These other agents can be administered to the cells expressing C3a receptor (C3aR) and C5a receptor (C5aR) and optionally at least one growth factor receptor (e.g., RTK) at amount effective to potentiate, enhance, promote, or increase cell growth, viability, and/or mitosis.

In another embodiment, the agent that promotes or stimulates C3aR and/or C5aR signaling of the cells can include C3, C5, C3a, C5a, a C3aR agonist, or C5aR agonist or an agent that causes, increases, and/or upregulates expression of at least one of C3, C5, C3a, C5a, a C3aR agonist, or C5aR agonist in or about the periphery of the wound. An example of a C3aR agonist include the linear peptide WWGKKYRASKLGLAR (SEQ ID NO: 3), which is described in Ember J A, et al., Biochemistry 1991, Apr. 16:30(15):3603-12. An example of a C5aR agonist is decapeptide analogue, Tyr-Ser-Phe-Lys-Pro-Met-Pro-Leu-DAla-Arg (SEQ ID NO: 4), that also binds to the C3a receptor, C3aR.

In some embodiments, the at least one of C3, C5, C3a, C5a, a C3aR agonist, or C5aR agonist can expressed in or about the periphery of the wound. For example, the at least one of C3, C5, C3a, C5a, a C3aR agonist, or C5aR agonist can be an expression product of a genetically modified cell. The target cells can include cells within or about the periphery of the wound or ex vivo cells that are biocompatible with the wound being treated. The biocompatible cells can also include autologous cells that are harvested from the subject being treated and/or biocompatible allogeneic or syngeneic cells, such as autologous, allogeneic, or syngeneic stem cells (e.g., mesenchymal stem cells), progenitor cells (e.g., multipotent adult progenitor cells) and/or other cells that are further differentiated and are biocompatible with the wound being treated.

The agent that increases, enhances, or promotes C3aR and/or C5aR signaling can be administered to the cells in vivo or in vitro. The cell can be derived from a human subject, from a known cell line, or from some other source. Examples of cells expressing C3a receptor (C3aR) and C5a receptor (C5aR) and optionally at least one growth factor receptor (e.g., RTK) include smooth muscle cells, endothelial cells, epithelial cells, or fibroblasts that are located in, for example, a tissue of a human subject.

“Administration”, as used herein, means provision or delivery of the agents that increase, at least one of C3aR and/or C5aR signaling in an amount(s) and for a period of time(s) effective to exert would healing and/or wound treating effects, such as growth, viability, and/or mitosis of cells proximate or about the periphery of the wound. Therapeutically effective doses of the agents that increase, promote, or enhance at least one of C3aR and/or C5aR signaling are readily determinable using data from an animal model.

The wounds treated by the method and/or agents can include any injury to any portion of the body of a subject (e.g., internal wound or external wound) including: acute conditions or wounds, such as thermal burns, chemical burns, radiation burns, burns caused by excess exposure to ultraviolet radiation (e.g., sunburn); damage to bodily tissues, such as the perineum as a result of labor and childbirth; injuries sustained during medical procedures, such as episiotomies; trauma-induced injuries, such as cuts, incisions, excoriations, injuries sustained as result of accidents, ulcers, such as pressure ulcers, diabetic ulcers, plaster ulcers, and decubitus ulcer, and post-surgical injuries. The wound can also include chronic conditions or wounds, such as pressure sores, bedsores, conditions related to diabetes and poor circulation, and all types of acne. In addition, the wound can include dermatitis, such as impetigo, intertrigo, folliculitis and eczema, wounds following dental surgery; periodontal disease; and tumor associated wounds.

In some embodiments, a method of promoting wound healing can include restoring wound healing in a subject where there has been a significant delay in wound healing. For example, it is often desirable to promote or increase the rate of healing in the case of both chronic wounds (such as diabetic, venous), acute (such as burns, penetrative injuries, or even wounds resulting from elective surgery), and for healing compromised individuals (such as immunodeficencies and the elderly). In all examples, the wounds, and a delay in healing of the wounds, can in the worst-case lead to death, but in general severely decrease the quality of life.

It will be appreciated that the present application is not limited to the preceding wounds or injuries and that other wounds or tissue injuries whether acute and/or chronic can be treated by the compositions and methods of the present invention.

In some aspects, the agent that increases, enhances, or promotes C3aR and/or C5aR signaling can be administered systemically to the subject or directly to or about the periphery of a wound. In one example, the period of time that the agent is administered to the wound and/or proximate the wound can comprise from about onset of the wound and/or tissue injury to about days, weeks, or months after tissue injury.

For example, the agent that promotes or stimulates C3aR and/or C5aR signaling of the cells can be delivered to or about the periphery of the wound by administering the agent neat or in a pharmaceutical composition to or about the wound. The pharmaceutical composition can provide localized release of the agent to the wound or cells being treated. Pharmaceutical compositions in accordance with the invention will generally include an amount of the agent that promotes or stimulates C3aR and/or C5aR signaling of the cells admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. Moreover, for human administration, preparations should meet sterility, pyrogenicity, general safety and purity standards as required by FDA Office of Biological Standards.

The pharmaceutical composition can be in a unit dosage injectable form (e.g., solution, suspension, and/or emulsion). Examples of pharmaceutical formulations that can be used for injection include sterile aqueous solutions or dispersions and sterile powders for reconstitution into sterile injectable solutions or dispersions. The carrier can be a solvent or dispersing medium containing, for example, water, ethanol, polyol (e.g., glycerol, propylene glycol, liquid polyethylene glycol, and the like), suitable mixtures thereof and vegetable oils.

Proper fluidity can be maintained, for example, by the use of a coating, such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the methods described herein in the required amount of the appropriate solvent with various amounts of the other ingredients, as desired.

Pharmaceutical “slow release” capsules or “sustained release” compositions or preparations may be used and are generally applicable. Slow release formulations are generally designed to give a constant drug level over an extended period and may be used to deliver the agent. The slow release formulations are typically implanted in the vicinity of the wound site, for example, at the site of a cell expressing C3aR and/or C5aR in or about the ischemic tissue.

Examples of sustained-release preparations include semipermeable matrices of solid hydrophobic polymers containing an agent that promotes or stimulates C3aR and/or C5aR signaling of the cells, which matrices are in the form of shaped articles, e.g., films or microcapsule. Examples of sustained-release matrices include polyesters; hydrogels, for example, poly(2-hydroxyethyl-methacrylate) or poly(vinylalcohol); polylactides, e.g., U.S. Pat. No. 3,773,919; copolymers of L-glutamic acid and γ ethyl-L-glutamate; non-degradable ethylene-vinyl acetate; degradable lactic acid-glycolic acid copolymers, such as the LUPRON DEPOT (injectable microspheres composed of lactic acid-glycolic acid copolymer and leuprolide acetate); and poly-D-(−)-3-hydroxybutyric acid.

While polymers such as ethylene-vinyl acetate and lactic acid-glycolic acid enable release of molecules for over 100 days, certain hydrogels release proteins for shorter time periods. When encapsulated agent remain in the body for a long time, and may denature or aggregate as a result of exposure to moisture at 37° C., thus reducing biological activity and/or changing immunogenicity. Rational strategies are available for stabilization depending on the mechanism involved. For example, if the aggregation mechanism involves intermolecular S—S bond formation through thio-disulfide interchange, stabilization is achieved by modifying sulfhydryl residues, lyophilizing from acidic solutions, controlling moisture content, using appropriate additives, developing specific polymer matrix compositions, and the like.

In certain embodiments, liposomes and/or nanoparticles may also be employed with the agent that promotes or stimulates C3aR and/or C5aR signaling of the cells. The formation and use of liposomes is generally known to those of skill in the art, as summarized below.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 \acute{Å}, containing an aqueous solution in the core.

Phospholipids can form a variety of structures other than liposomes when dispersed in water, depending on the molar ratio of lipid to water. At low ratios, the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via four different mechanisms: Endocytosis by phagocytic cells of the reticuloendothelial system, such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the methods described herein, and such particles may be are easily made.

In some embodiments, an agent that increases, enhances, or promotes at least one of C3aR and/or C5aR signaling can be formulated for topical administration through the skin. “Topical delivery systems” also include transdermal patches containing the ingredient to be administered. Delivery through the skin can further be achieved by iontophoresis or electrotransport, if desired.

Formulations for topical administration to the skin include, for example, ointments, creams, gels and pastes comprising the complement antagonist in a pharmaceutical acceptable carrier. The formulation of agents for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

In some embodiments, he agent that promotes or stimulates C3aR and/or C5aR signaling can be provided in and/or on a substrate, solid support, and/or wound dressing for delivery to the wound. As used herein, the term “substrate,” or “solid support” and “wound dressing” refer broadly to any substrate when prepared for, and applied to, a wound for protection, absorbance, drainage, etc. The substrate may include any one of the numerous types of substrates and/or backings that are commercially available, including films (e.g., polyurethane films), hydrocolloids (hydrophilic colloidal particles bound to polyurethane foam), hydrogels (cross-linked polymers containing about at least 60% water), foams (hydrophilic or hydrophobic), calcium alginates (non-woven composites of fibers from calcium alginate), and cellophane (cellulose with a plasticizer). The shape and size of a wound may be determined and the wound dressing customized for the exact site based on the measurements provided for the wound. As wound sites can vary in terms of mechanical strength, thickness, sensitivity, etc., the substrate can be molded to specifically address the mechanical and/or other needs of the site. For example, the thickness of the substrate may be minimized for locations that are highly innervated, e.g., the fingertips. Other wound sites, e.g., fingers, ankles, knees, elbows and the like, may be exposed to higher mechanical stress and require multiple layers of the substrate.

The agent that promotes or stimulates C3aR and/or C5aR signaling can also be provided in or on a surface of a medical device used to treat an internal and/or external wound. The medical device can comprise any instrument, implement, machine, contrivance, implant, or other similar or related article, including a component or part, or accessory, which is, for example, recognized in the official U.S. National Formulary, the U.S. Pharmacopoeia, or any supplement thereof; is intended for use in the diagnosis of disease or other conditions, or in the cure, mitigation, treatment, or prevention of disease, in humans or in other animals; or, is intended to affect the structure or any function of the body of humans or other animals, and which does not achieve any of its primary intended purposes through chemical action within or on the body of man or other animals, and which is not dependent upon being metabolized for the achievement of any of its primary intended purposes.

The medical device can include, for example, endovascular medical devices, such as intracoronary medical devices. The medical device may additionally include either implantable pacemakers or defibrillators, vascular grafts, sphincter devices, urethral devices, bladder devices, renal devices, gastroenteral and anastomotic devices, vertebral disks, hemostatic barriers, clamps, surgical staples/sutures/screws/plates/wires/clips, glucose sensors, blood oxygenator tubing, blood oxygenator membranes, blood bags, birth control/IUDs and associated pregnancy control devices, cartilage repair devices, orthopedic fracture repairs, tissue scaffolds, CSF shunts, dental fracture repair devices, intravitreal drug delivery devices, nerve regeneration conduits, electrostimulation leads, spinal/orthopedic repair devices, wound dressings, embolic protection filters, abdominal aortic aneurysm grafts and devices, neuroaneurysm treatment coils, hemodialysis devices, uterine bleeding patches, anastomotic closures, aneurysm exclusion devices, neuropatches, vena cava filters, urinary dilators, endoscopic surgical and wound drainings, surgical tissue extractors, transition sheaths and dialators, coronary and peripheral guidewires, circulatory support systems, tympanostomy vent tubes, cerebro-spinal fluid shunts, defibrillator leads, percutaneous closure devices, drainage tubes, bronchial tubes, vascular coils, vascular protection devices, vascular intervention devices including vascular filters and distal support devices and emboli filter/entrapment aids, AV access grafts, surgical tampons, cardiac valves, and tissue engineered constructs, such as bone grafts and skin grafts.

In other embodiments, the agent that promotes or stimulates C3aR and/or C5aR signaling of the cells can be administered in combination with a growth factor that promotes wound repair and/or mitigates apoptosis of cells of the wound. The growth factor can include, for example, VEGF, NGF, GM-SCF, EGF, FGF, IGF, BDNF, BMP, SDF-1, and/or HGF.

The following examples are for the purpose of illustration only and are not intended to limit the scope of the claims, which are appended hereto.

### Example 1

Complement has been shown to be an important component in various pathological responses involving endothelial cells (ECs), in all cases the effects have been attributed to serum complement. We tested whether autocrine GPCR signal transduction might be connected with the anti-apoptotic and/or mitotic effects of VEGF on ECs.

### Materials and Methods

### Reagents and Antibodies

VEGF-A was from Prospect (Ness Ziona, Israel). C3aR antagonist (C3aR-A) and C5aR antagonist (C5aR-A) are from Merck (Darmstadt, Germany). Anti-C3 and Anti-05 was purchased from BD PharMingen (San Diego, Calif.). Anti-C3aR and Anti-05aR were purchased from Santa Cruz Biotech (Santa Cruz, Calif.). Endothelial cell growth supplement was purchased from BD Biosciences (San Diego, Calif.). Anti-phospho-VEGFR2 (pYpY1054/1059) was purchased from Invitrogen (Camarillo, Calif.).

### Cells & the Isolation of Primary Aortic ECs

bEnd.3 cells and MS1 murine EC lines were cultured in 10% and 5% FBS, respectively with DMEM. The primary mouse aorta endothelial cells (MAECs) were isolated from wild-type C57BL/6J mice (WT), Daf1^{-/-}, C3aR^{-/-}C5aR^{-/-} at ages from 1 to 4 months, by utilizing a non-mechanical and non-enzymatic method. The outgrowth of endothelial cells from aortic rings was markedly facilitated within first 72 h in the absence of antibiotics. Aortic rings thus were discarded at culture day 3 to avoid the possible contamination of non-endothelial cell types. After removing the aortic rings, cells were maintained in completed DMEM/F12 medium consisting of 20% FBS, 2 mM L-glutamine, 1 nonessential amino acid, 0.05 mg/ml endothelial cell growth supplement (ECGS), 100 units/ml penicillin, 100 g/ml streptomycin, and 0.1 mg/ml heparin until confluent.

### Gene Expression and Flow Cytometry

RNA was isolated by the TRIzol method (Invitrogen). Reverse transcription was achieved with Superscript-III reverse transcriptase (Invitrogen) using supplied oligo dT primers. qPCR was performed in a 24 μl volume with SYBR Green PCR mix (Applied Biosystems) using gene specific qPCR primers.

For C3aR and C5aR staining cells were harvested after plating in 10% FBS supplemented DMEM via Versene (Invitrogen) and stained by using three-layer immunoenzyme method. Stained cells were analyzed on a Becton Dickinson LSRII.

### Quantitation of Cell Growth

For studies with bEnd.3 and MS-1 cells, 5×10⁴ cells were plated in 24-well plates and allowed to adhere for 24 hr. Following culturing in 0.5% FBS with DMEM for another 24 hours, the cells were treated as described in the Fig legends. Growth was quantified manually at 24, 48 and 72 hr with trypan blue. At least 95% of cells were viable in all experiments. VEGF-A was used at a concentration of 30 ng/ml, C3a and C5a at 1 ug/ml, and anti-C3 and ant-C3 mAbs at I ug/ml.

### C3a/C5a/VEGF ELISAs and Propidium Iodide Staining

Enzyme-linked immune-adsorbent assays were conducted to quantify C3a and C5a levels in culture supernatants. Ninety six well plates were used and the manufacturer's (eBioscience) protocol followed. FACS without sodium azide was used for diluents with blocking buffer and color was developed using enhanced TMB solution with H₂O₂. The stop solution consisted of 2 N H₂SO₄.

Propidium iodide staining was performed. In brief, the cells were serum starved for 24 hrs followed by administration of 1 μM colchicine (Sigma). 16 hrs following colchicine treatment, cells were given either growth factor treatment or 17 ng/mL mC5a. After an additional 24 hrs, cells were removed from plating via Trypsin/EDTA and fixed in 0.25% Formaldehyde for 10 mins at 37° C. Cells were spun out of formaldehyde solution and resuspended in 90% methanol at 4° C. until assayed. Following removal from methanol, excess RNA was removed via treatment with RNase (Sigma) and stained with propidium iodide for 30 mins at 4° C. Cells were analyzed on an Epics XL.

### Hypoxia and 2-D HUVEC Tube Formation

The mitochondrial uncoupler protonophore carbonyl cyanide p-(trifluoromethoxy)phenylhydrazone (FCCP)+iodoacetate (IAA), which simulate hypoxia when added to cells, was incubated for 1 hr and 2 hr with HUVEC, and C3, fB, and fD mRNA levels were assayed by qPCR.

Fifty μL of the growth factor reduced matrigel (BD Biosciences) was allowed to polymerize in 96-well plate at 37° C. for 30 minutes. Triplicates of 25,000 HUVEC were plated onto the prepared matrigel in a volume of 150 μL of EBM-2 media (Lonza) in four different conditions: no growth factors, 30 ng/ml of VEGF, 30 ng/ml of VEGF and with 15 ng/ml each of C3aR-A and C5aR-A and the complete set of factors. After approximately 15 hr, images were captured using a light microscope in high magnification.

### Corneal Neovascularization and Tumor Angiogenesis Model

Corneal neovascularization was induced in 6-8 wk-old (C57BL/6) WT, Daf1^{-/-}, C3aR^{-/-} C5aR^{-/-}, and Daf1^{-/-}C3aR^{-/-}C5aR^{-/-} mice (n=5 each group) by placing a non-penetrating suture (0-11 Nylon, Alcon Inc.) in the center of the cornea under a dissecting microscope. On days 7, 9, 11, and 15, corneal vessels were examined after intravenous administration of 100 μl of 2.5% fluorescein-dextran (Sigma) by fluorescence microscopy. The protocol was approved by the Case Western Reserve University Institutional Animal Care and Use Center (IACUC). Male mice were injected subcutaneously with 1.5×10⁶RM1 prostate cancer cells. Tumors were collected 10 days after injection and were weighed, photographed, snap-frozen in OCT and processed for immunohistochemical staining with biotin-conjugated rat anti-mouse CD31 antibody (BD Biosciences, San Jose, Calif.). Stained sections were analyzed using fluorescent or bright field imaging microscopy (Leica, Germany) and ImagePro Plus Capture and Analysis software version 6.1 (Media Cybernetics). CD31-positive areas were quantified in 5-10 independent fields per tumor implant.

### Results

ECs Locally Synthesize Complement, C3a/C5a are Generated from this Local Synthesis, and Autocrine Interactions of these Anaphylatoxins with C3aR/C5aR on ECs are Essential to Sustain EC Viability

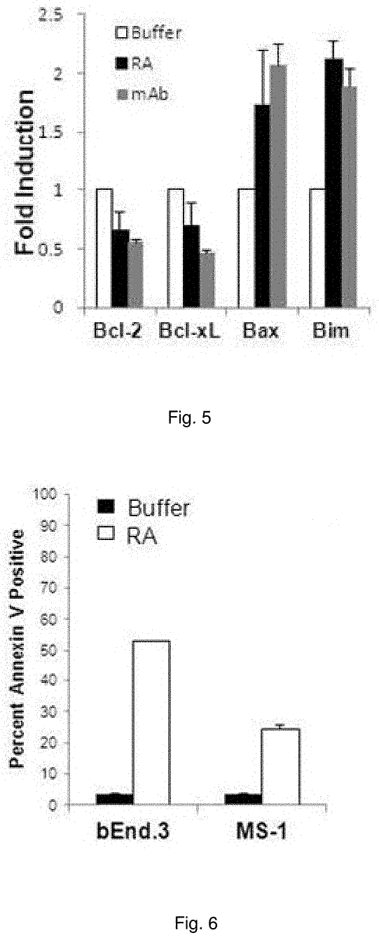

As a first test of whether ECs, like CD4⁺ tonically synthesize complement (under homeostatic conditions) and whether this local synthesis leads to autocrine C3aR/C5aR signaling in ECs, we examined serum free cultures of bEnd.3 and MS-1 murine EC cell lines for local C3a/C5a production and for surface expression of C3aR/C5aR. The culture supernatants of both EC lines contained C3a and C5a and both EC lines expressed both GPCRs (FIG. 4) To test whether tonic C3aR/C5aR signaling provides survival signals to ECs as found for CD4⁺ cells, we added pharmacological C3aR/C5aR antagonists (C3aR-A/C5aR-A) or with anti-C3a/C5a mAbs specific for neo-epitopes in the C3a/C5a ligands (not present on the parental C3/C5 proteins) to the serum free cultures and assayed for markers of apoptosis. Blockade of either the receptors or their ligands evoked surface Fas/FasL expression, caused decreased intracellular Bcl-2/Bcl-x2 and increased Bax/Bim mRNAs, and rendered the ECs susceptible to Annexin V binding (FIGS. 5-6). The effects of the C3aR/C5aR blockade on Bcl-2 mRNA expression in ECs simulated that of blockade of VEGFR2 signaling.

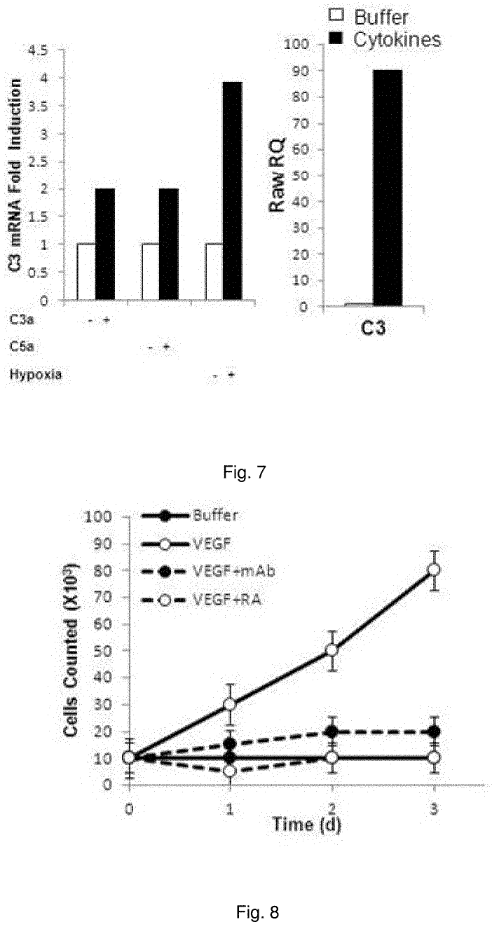

We next conversely assessed how transducing C3aR/C5aR signals into ECs affects endogenous EC complement production and whether it is connected with “classical” EC responses. We incubated HUVEC with 1) inflammatory cytokines, 2) the mitochondrial uncoupler protonophore carbonyl cyanide p-(trifluoromethoxy)phenylhydrazone (FCCP)+iodoacetate (IAA) (which mimicks hypoxia), or 3) C3a or C5a, after which we assayed complement mRNA transcripts by qPCR. The mitochondrial uncoupler evoked an increase in complement transcripts consistent with this signal transduction functioning to amplify EC growth and prevent apoptosis in response to hypoxia (FIG. 7). Added IL-1/IFN-γ/TNF-α had the same effect (FIG. 7). Both added C3a and added C5a increased transcription of C3, indicative of the ability of both anaphylatoxins to establish auto-inductive signaling loops (FIG. 8).

### VEGF Growth Induction Depends on C3aR/C5aR Signaling

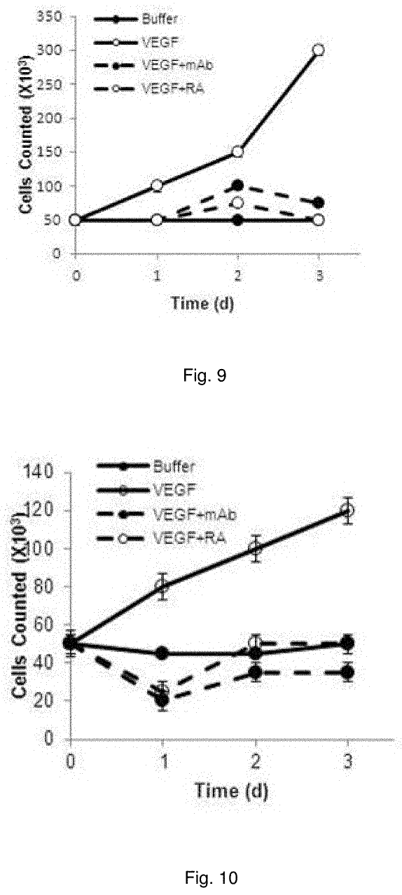

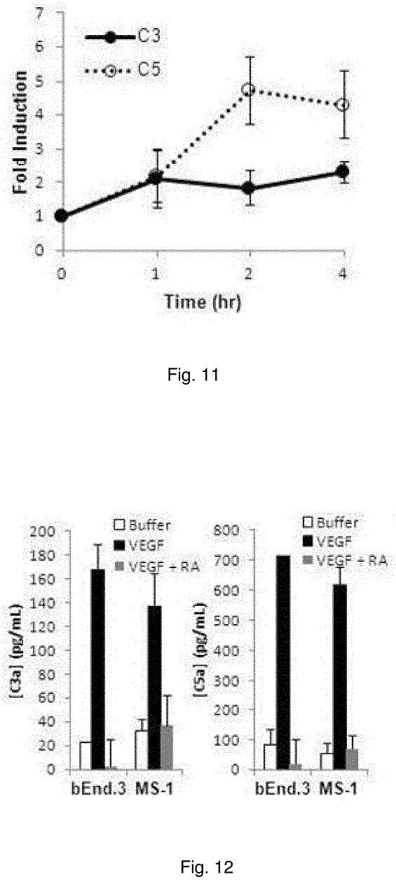

As a first test of whether VEGF growth induction and autocrine C3aR/C5aR signaling in ECs are mechanistically connected, we added VEGF-A to serum starved MS-1 and bEnd⋅3 cells in the absence or presence of C3aR-A/C5aR-A or anti C3a/C5a mAbs and counted cell numbers over 72 hr. Blockade of either the receptors or their C3a/C5a ligands abolished VEGF-A induced proliferation of both EC lines (FIGS. 8-9). A similar effect was observed in HUVEC cultures (FIG. 10). Blockade of the individual receptors or ligands individually (data not shown) inhibited VEGF-A induced growth and that blockade of both synergized in the inhibition. C3aR/C5aR antagonism or C3a/C5a neutralization did not affect the growth of CTLL cells which is IL-2 dependent and only partially inhibited that of MS-1 or bEnd⋅3 in 10% plasma verifying that the inhibitory effects on VEGF-A growth induction were specific rather toxic effects on the cells. To determine whether VEGF growth induction is interconnected with upregulated C3aR/C5aR signaling, we examined the effect of added VEGF-A on local complement production by MS-1 and bEnd⋅3 cells. qPCR of cell extracts and ELISAs of their culture supernatants showed that VEGF-A both up-regulated C3/C5 mRNA transcripts and increased local C3a/C5a production (FIGS. 11-12). These findings show that amplified C3aR/C5aR GPCR signals integrate with VEGF growth signals.

Because the disruptive effect of C3aR/C5aR antagonism on VEGF growth induction could be a consequence of the requirement of autocrine C3aR/C5aR signaling for EC viability, we performed cell cycle assays. Adding C5a to serum starved HUVEC caused transition from G0 into G2 identically to adding VEGF-A. Moreover, C3aR/C5aR blockade prevented VEGF-A triggering of the cell cycle. These findings that autocrine C3aR/C5aR signaling not only prevents EC apoptosis but drives EC growth point to a bidirectional signaling mechanism wherein C3aR/C5aR GPCR signaling transactivates to VEGFR2 signaling and vice versa.

### C3aR/C5aR Signals are Required for VEGF Induced Growth of ECs

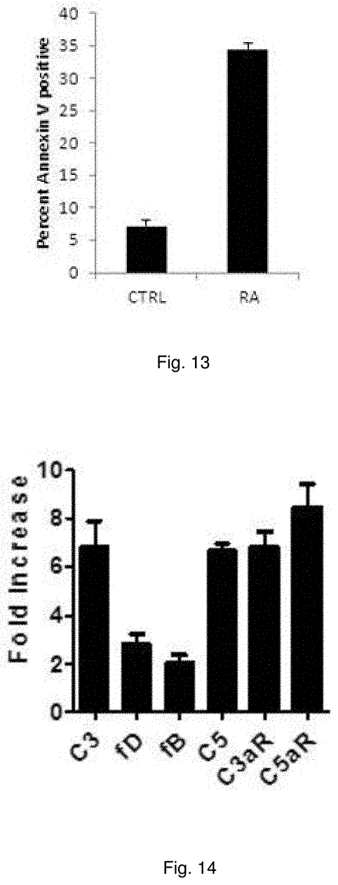

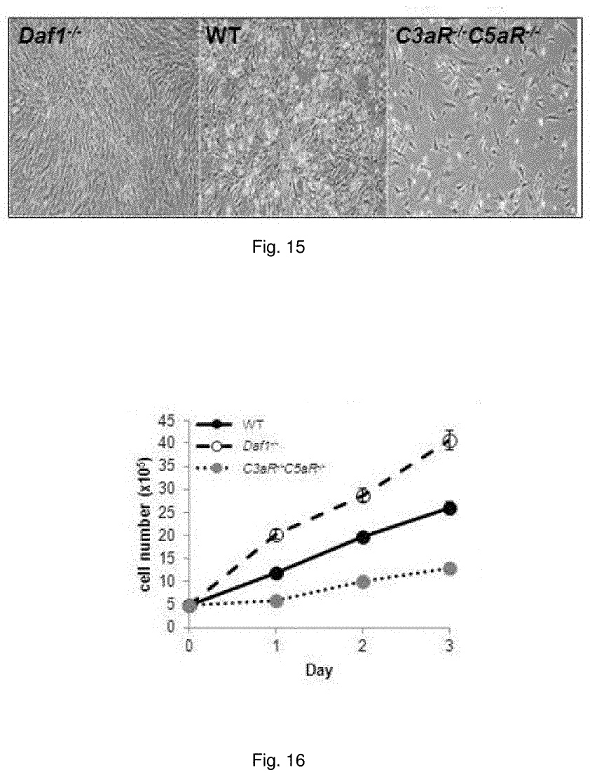

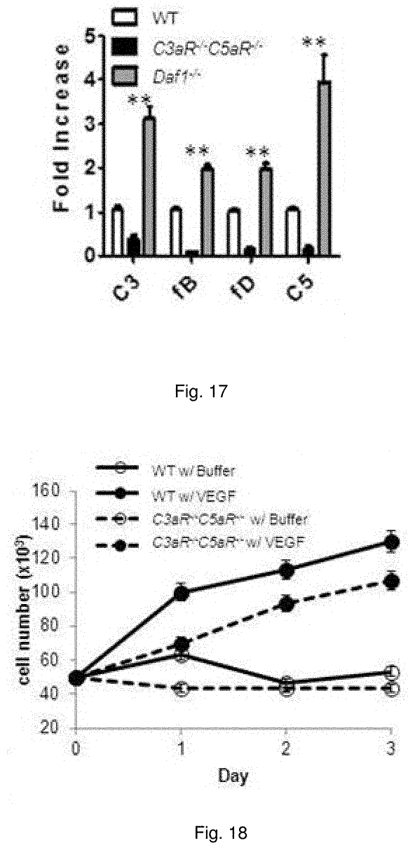

To validate that the above findings apply physiologically, we prepared primary ECs from aortas of WT mice. As found for the bEND.3 and MS-1 EC lines and for HUVEC, aortic ECs from WT mice expressed C3aR and C5aR, antagonizing C3aR/C5aR signaling induced markers of apoptosis (FIG. 13), and VEGF-A upregulated mRNA transcripts of all of the components/receptors associated with autocrine C3aR/C5aR signaling (FIG. 14). We similarly prepared aortic ECs from mice in which C3aR/C5aR signaling is potentiated (Daf1^{-/-} mice) or C3aR and C5aR are deleted (C3aR^{-/-}C5aR^{-/-} mice). Side by side comparisons of the growth rates of the Daf1^{-/-} and C3aR^{-/-}C5aR^{-/-} aortic ECs to that of WT aortic ECs (FIG. 15) showed that the absence of DAF favored EC proliferation (1.77±0.06 vs 1.30±0.017×10⁵ cells 24 hr after plating 10⁵ cells) whereas the absence of C3aR/C5aR virtually abolished it (1.07±0.05×10⁵ cells). Quantitative studies in which we cultured 5×10⁴ ECs from the three genotypes over a 72 hr period and counted cell numbers documented an ˜2-fold faster growth (2×10⁶±0.15 vs 4×10⁶±0.017 p<0.005) of Daf1^{-/-} ECs than of WT ECs (consistent with potentiated C3aR/C5aR signaling), and little or no growth (2×10⁶±0.15 vs 1×10⁶±0.05p<0.005) of C3aR^{-/-}C5aR^{-/-} ECs (consistent with precluded C3aR/C5aR signaling) (FIG. 16). To establish if the differences in growth rates correlate with differences in EC complement production in vivo, we assayed perfused aortas from Daf1^{-/-}, WT, and C3aR^{-/-}C5aR^{-/-} mice for mRNA transcripts of complement components connected with C3aR/C5aR signaling. qPCR assays documented upregulated and suppressed C3/fB/fD/C5 mRNA transcripts in Daf1^{-/-} and C3aR^{-/-}C5aR^{-/-} aortas, respectively (FIG. 17). To study how the absence of C3aR/C5aR signaling affects VEGF growth induction, we serum starved aortic ECs from WT and C3aR^{-/-}C5aR^{-/-} mice and added VEGF-A to cultures. The genetic absence of C3aR/C5aR markedly suppressed VEGF induced growth (FIG. 18). However, unlike the virtual abrogating effects of disrupted C3aR/C5aR signaling in WT ECs with the antagonists or mAbs, VEGF-A retained some growth inductive capacity.

### C3aR/C5aR Signaling in ECs is Essential for Vascular Tube Formation and for Angiogenesis In Vivo

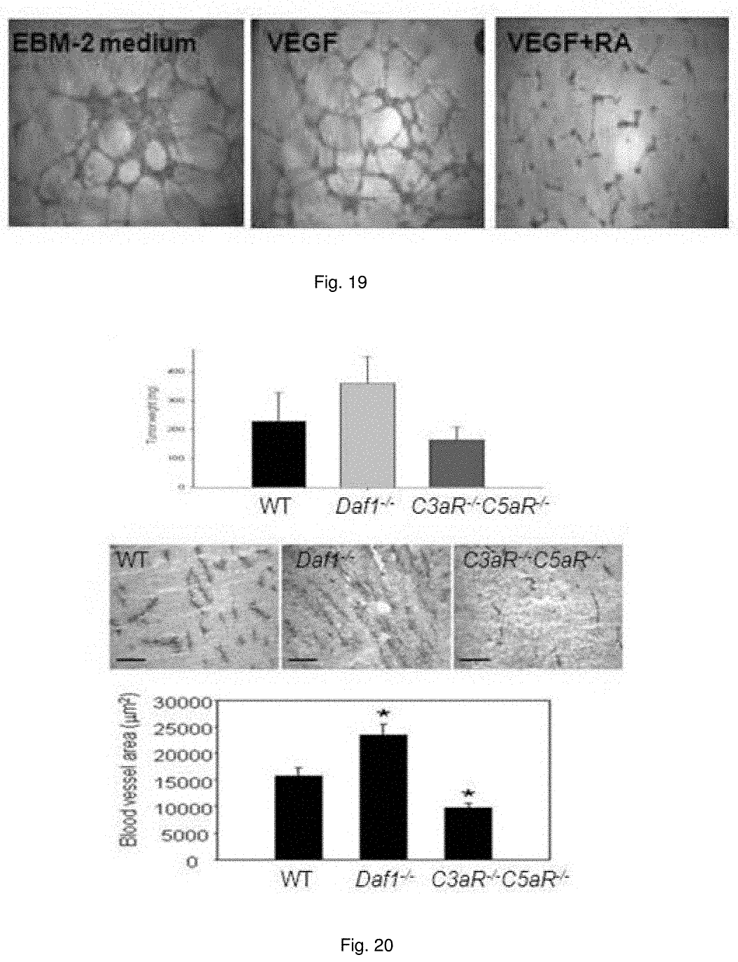

As a first test of whether the above connection of VEGF growth induction with autocrine C3aR/C5aR signaling is physiologically relevant, we performed two-dimensional tube formation assays with HUVEC to determine how this GPCR signaling affects angiogenesis. Culturing HUVEC under conventional conditions with EBM-2 basal medium containing supplemental growth factors yielded the typical growth pattern of an EC tubal network consisting of cluster regions with a few braches. (FIG. 19, Left). Substitution of exogenous VEGF for the growth factor cocktail evoked increased branch points (FIG. 19, Middle). In contrast, the inclusion of C3aR-A/C5aR-A together with VEGF-A under identical conditions essentially disrupted tube formation (FIG. 19, Right).

To establish whether in vivo angiogenesis, in fact, depends on C3aR/C5aR signaling, we used two in vivo models. In the first model, we placed non-penetrating sutures in the corneas of WT, Daf1^{-/-}, C3aR^{-/-}C5aR^{-/-} and Daf1^{-/-}C3aR^{-/-}5aR^{-/-} mice. This assay has the advantage of measuring neo-angiogenesis (new blood vessel growth) since the cornea is normally avascular. Beginning at day 5, we examined corneas by fluorescence confocal microscopy after intravenously administering fluorescent dextran beads. In contrast to the gradual influx of blood vessels at the corneal margins starting at day 9 in WT mice, markedly accelerated and more robust influx occurred in Daf1^{-/-} mice, and virtually none occurred in C3aR^{-/-}C5aR^{-/-} or Daf1^{-/-}C3aR^{-/-}C5aR^{-/-} mice.

In the second model, we implanted RM1 prostate tumors which are highly vascularized and dependent on angiogenesis for their progression into the flanks of male WT, Daf1^{-/-} and C3aR^{-/-}C5aR^{-/-} mice. We harvested the tumors at day 14 post inoculation, weighed them, and immunohistochemically examined sections of their stroma staining for the EC marker CD31. The tumors in C3aR^{-/-}C5aR^{-/-} mice were smaller (227.93±201.28 vs 164.33±89.7, p=0.29) than in WT mice, and larger (227.93±201.28 vs 359±185.31, p=0.18) than in Daf1^{-/-} mice (FIG. 20, Top). The sections showed significantly reduced vessel density and vascular area in tumors grown in C3aR^{-/-}C5aR^{-/-} mice compared to WT mice (9772±799 vs. 15699±1591 μm², P=0.004, n=8) (FIG. 20, Bottom). In contrast, angiogenesis was significantly enhanced in tumors from Daf1^{-/-} mice (23458±1976 vs. 15699±1591 μm², P=0.01, n=8).

### C3aR/C5aR Signaling is Essential for VEGFR2 Phosphorylation

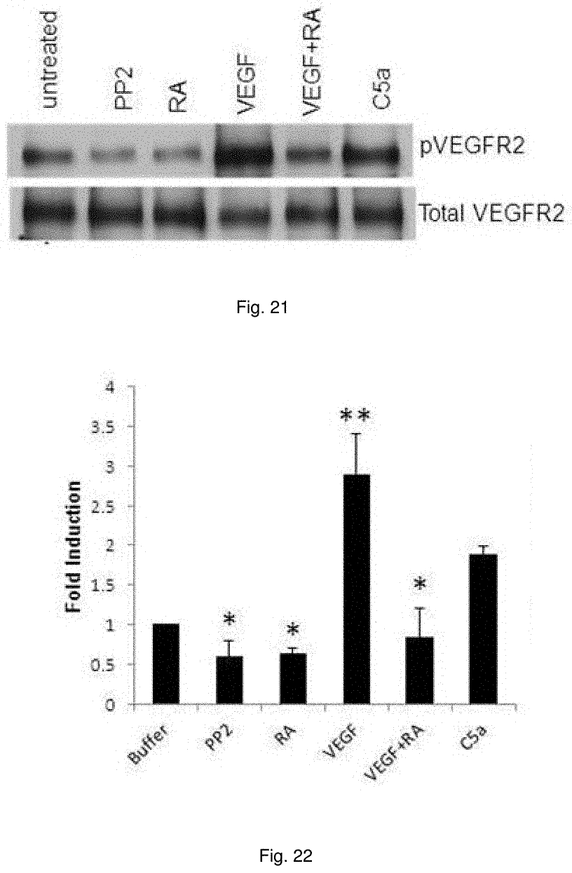

To directly establish whether C3aR/C5aR signaling is integral to VEGF signaling, we next evaluated phosphorylation of VEGFR2. We tested 1) how adding C5a to primary WT ECs affects VEGFR2 autophosphorylation and 2) whether VEGFR2 phosphorylation induced by added VEGF-A is affected by antagonizing C3aR/C5aR signaling. We incubated serum starved WT aortic ECs with C5a or with VEGF-A in the absence or presence of C3aR-A/C5aR-A, after which we probed immunoblots of protein lysates and anti-pVEGFR2^{Y1054/1059} mAb or pan VEGFR2 antibody. The added C5a increased VEGFR2^{Y1054/1059} phosphorylation relative to untreated controls and the C3aR/C5aR blockade virtually abolished VEGF-A induced VEGFR2 auto-phosphorylation (FIG. 21-22).

Up-Regulation of VEGFR2 and Endogenous VEGF-A Production Compensates for Pro-Apoptotic Signaling in C3aR^{-/-}C5aR^{-/-} ECs

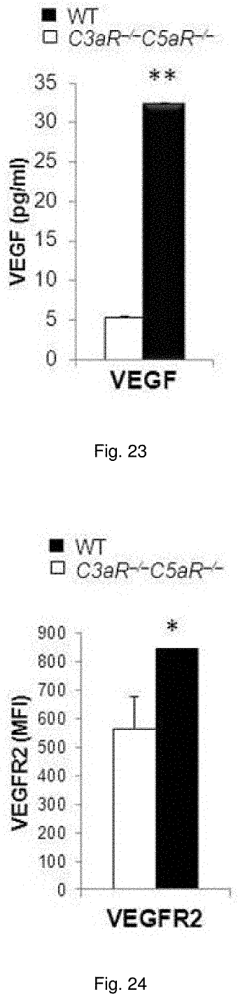

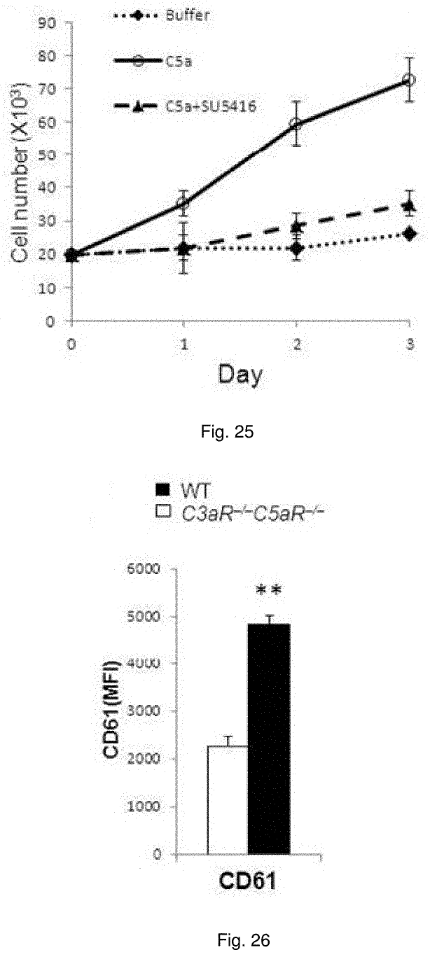

The above experiments with WT ECs in this Example showed that autocrine C3aR/C5aR signaling provides survival signals and that blockade of this signaling triggers both the intrinsic and extrinsic PCD pathways. We compared VEGFR2 expression levels and endogenous VEGF-A production in C3aR^{-/-}C5aR^{-/-} and WT ECs. ELISAs of their supernatants of untreated the C3aR^{-/-}C5aR^{-/-} ECs (FIG. 23) showed 5-fold more VEGF-A production compared to WT ECs, and flow cytometry (FIG. 24) showed 2-fold increased levels of VEGFR2 on their surfaces. Addition of the VEGFR2 inhibitor SU5416 to cultures of C3aR^{-/-}C5aR^{-/-} ECs without or with added C5a (FIG. 25) abolished their growth, indicating that upregulated VEGFR2 auto-phosphorylation compensated for the loss of C3aR/C5aR signaling in the knockouts and that EC viability depended entirely on this compensation. Because β3 Integrin (CD61) has been reported to be regulated by VEGFR2 signaling and implicated in EC survival signaling, we compared CD61 expression in C3aR^{-/-}C5aR^{-/-} and WT ECs. Flow cytometry analysis of C3aR^{-/-} C5aR^{-/-} ECs showed 2.5-fold increased CD61 levels (FIG. 26).

### VEGF Induces C3aR/C5aR Signaling by an IL-6 and Stat3 Dependent Mechanism

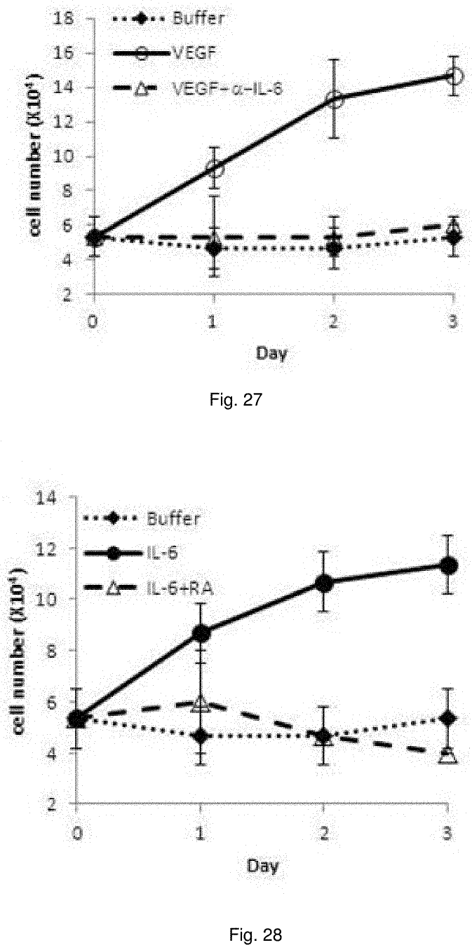

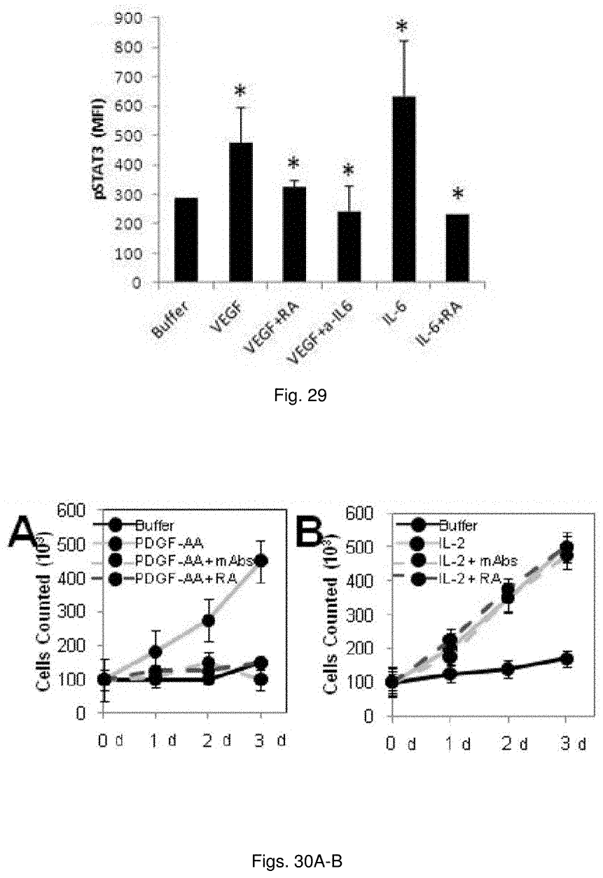

While the experiments above showed that C3aR/C5aR signaling transactivates to phosphorylate VEGFR2, they left unanswered how VEGFR2 signaling is mechanistically linked to C3aR/C5aR signaling. Numerous past studies have linked VEGF with both IL-6 and p-Stat3 our prior studies by ourselves has shown that C5a induces IL-6 and that IL-6 signaling is interconnected with C3aR/C5aR signaling. To determine if the linkage between VEGF and C3aR/C5aR signaling in ECs involves IL-6, we incubated MS-1 ECs with 1) VEGF-A alone, IL-6 alone, or VEGF-A plus anti-IL-6 mAb, or with 2) IL-6 alone or IL-6 plus C3aR-A/C5aR-A, and assayed cell growth. Growth induction by IL-6 induced EC growth comparably to VEGF-A and VEGF-A's growth induction was abolished by anti-IL-6 (FIG. 27). The growth induction of ECs of IL-6, like that of VEGF-A, was abolished by C3aR-A/C5aR-A (FIG. 28). Both VEGF-A and IL-6 induced Stat3 phosphorylation and the Stat3 phosphorylation in both cases was abolished by C3aR/C5aR antagonism (FIG. 29). VEGF-A treatment or WT aortic ECs upregulated C3/C5 and increased local C3a/C5a generation as found in FIGS. 11-12 for MS-1 ECs, but neither change occurred in the presence of anti-IL-6 mAb or the JAK1 inhibitor. These finding thus indicate that VEGF interconnects with C3aR/C5aR signaling via the induction of IL-6 and its activation of Stat3.

The data show that targeting C3aR and C5aR can repress pre cancer or cancer associated angiogenesis. Based on the data in this study mechanistically connecting C3aR/C5aR signaling with VEGFR2 signaling, such targeting should have value in other conditions including diabetic retinopathy rheumatoid arthritis (RA) where it has been shown that hypoxia and consequent angiogenesis augments the proliferation of synovial fibroblasts, systemic sclerosis where it has been shown that VEGF induced EC growth contributes to uncontrolled fibroblast growth and external ear canal cholesteatoma (EACC), an invasive and destructive external otitis, where it has been shown that migration of keritinocytes into the external ear is connected with increased expression of VEGF in all layers of the EACC-epithelium.

### Example 2

Inflammatory cell influx and vascular cell proliferation underlie atherosclerotic progression and set the stage for thrombosis which eventuates in myocardial infarction and stroke. While many cell surface molecules and inflammatory mediators have been implicated in this self perpetuating process, the mechanisms that drive inflammation and proliferation remain incompletely characterized. We have found that both smooth muscle cells (SMCs) and monocytes/macrophages (mΦs) locally produce C3a and C5a activation fragments and that these anaphylatoxins interact with upregulated C3a and C5a receptors (C3aR and C5aR) on SMCs/mΦs. Our studies show that amplification of this signal transduction is what drives SMC/mΦ proliferation and evokes mΦs inflammatory cytokine production. This insight derived from our studies of immune cell activation which uncovered the previously unrecognized fact that local complement synthesis by interacting dendritic cells (DCs)- and T cells is an early event in T cell activation and that the resulting C3a/C5a-C3aR/C5aR interactions play a requisite role in T cell proliferation and effector cytokine, e.g., IFN-γ/IL-17 production. These studies in immune cells further showed that C3aR/C5aR signaling operates tonically to maintain T cell viability and suppress costimulatory molecule and innate cytokine, e.g., IL-1β/IL-12/IL-23 production. Importantly, our studies in SMCs/mΦs show that C3aR/C5aR signal transduction functions tonically similarly to that in immune cells.

Our work has shown that the generation of C3a/C5a from locally synthesized complement by SMCs/mΦs, like that from immune cells, is regulated by the cell surface C3/C5 convertase inhibitor DAF and our studies now show that DAF expression is controlled by Kruppel-like factor 4 (KLF4). We have found that C3aR/C5aR signaling drives the neointimal response to endothelial cell (EC) injury. Our studies show that the mechanism underlying this is that amplified C3aR/C5aR signal transduction is essential for growth induction by platelet derived growth factor (PDGF), a factor important in both the EC response to injury and atherogenesis. In this Example, we 1) further characterized the interconnections of C3aR/C5aR signal transduction with platelet, leukocyte, EC, and SMC responses to EC injury, and found 2) the connections of C3aR/C5aR signal transduction with atherogenesis and thrombosis.

We found most cell types locally synthesize complement, and this local complement synthesis controls many cellular responses. Of particular interest, are findings documenting the local generation of C3a/C5a and the interaction of these fragments with C3aR/C5aR on endothelial cells (ECs), smooth muscle cells (SMCs), as well as myeloid cells, all of which are relevant to atherogenesis and possibly to its thrombotic sequelae. Local synthesis and activation in an autocrine fashion sustains EC/SMC viability and alters cellular production of and/or response to growth factors and cytokines. Importantly, this signaling loop is regulated by the cell surface C3/C5 convertase inhibitor, decay accelerating factor (DAF or CD55). Downregulation of DAF potentiates while upregulation suppresses C3aR/C5aR signaling and attendant effects on cellular viability and activation.

We found that vascular inflammation and neointimal formation are potentiated in mice deficient in Daf1 (the murine homolog of the human DAF gene), but completely attenuated in Daf1^{-/-}C3aR^{-/-} and Daf1^{-/-}C5aR^{-/-} mice. Data indicate that hypoxia and pro-inflammatory cytokines upregulate local EC complement production and local C3a/C5a generation, promoting leukocyte recruitment and activation. Finally, we have found that DAF expression is regulated by Kruppel like factor 4 (KLF4) in both ECs and immune cells. Based on these newly uncovered interconnections of autocrine C3aR/C5aR signaling in ECs, SMCs, and leukocytes, we show that local complement synthesis and autocrine C3aR/C5aR signaling regulates vascular cell responses to injury, which in turn contributes the development of atherosclerosis and thrombosis.

We found that autocrine/paracrine C3aR/C5aR signaling in ECs/monocytes/mΦN/SMCs is mechanistically linked to key proliferative and inflammatory processes that underlie the neointimal response of ECs to injury and participate in atherosclerotic progression to thrombosis. Evidence below implicates local C3aR/C5aR signaling in vascular proliferation, inflammation, injury, and repair.

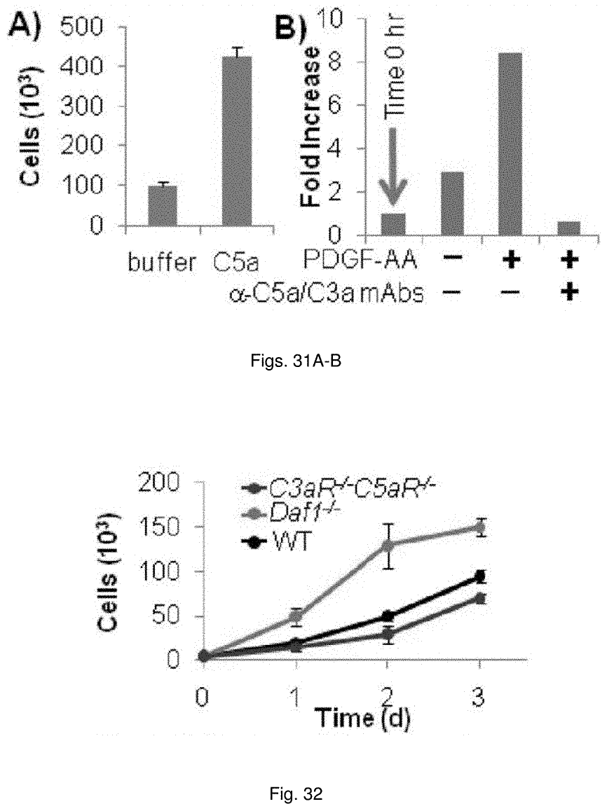

PDGF contributes to atherogenesis and plays a central role in neointimal proliferation following EC injury. In view of the growth difference of primary cultured Daf1^{-/-}, WT, C3aR^{-/-} C5aR^{-/-}, and C3^{-/-}C5^{-/-} SMCs, we examined whether C3aR/C5aR signals are interconnected with PDGF growth induction. For these studies, we utilized NIH-3T3 fibroblasts (which express PDGFR-αα) in conjunction with PDGF-AA. Following determination of optimal PDGF-AA doses, we incubated 1×10⁵ cells/well in triplicate with 30 ng/ml of PDGF-AA ±10 ng/ml of C3aR and C5aR antagonists (C3aR-A/C5aR-A), 10 ng/ml of anti-C3a/anti-C5a mAbs or respective controls, after which we quantified cell growth by counting cell numbers over time. Remarkably, blockade of either the receptors or their C3a/C5a ligands near completely suppressed PDGF-AA induced proliferation (FIG. 30A). Consistent with specificity, the growth of CTLL cells which is IL-2 dependent was not affected by C3aR/C5aR antagonism (FIG. 30B) and that of NIH-3T3 cells in 10% plasma was only partially inhibited. To directly establish whether C5aR signals augment PDGF-AA growth signaling, we 1) quantified the effect of added C5a one growth, and 2) assayed C5a in culture supernatants of PDGF-AA treated cells. This showed that a) added C5a (FIG. 31A) by itself induced proliferation, b) NIH-3T3 cells tonically produce C5a, and c) C5a generation by NIH-3T3 cells is amplified by PDGF-AA (FIG. 31B). Because the dependence of PDGF growth induction on C3aR/C5aR signaling could be indirect, i.e., a consequence of its requirement for viability, we performed cell cycle assays. Adding C5a to serum starved NIH-3T3 cells caused transition from G0 into G2 identically to adding PDGF indicating that autocrine C3aR/C5aR signals not only prevent apoptosis but drive growth.

To validate that these findings apply physiologically, we repeated the PDGF studies with the primary cultures of SMCs from aortas of WT, Daf1^{-/-}, and C3aR^{-/-}C5aR^{-/-} mice. Dat1^{-/-} SMCs (in which C3aR/C5aR signaling is potentiated) showed greater proliferation than WT SMCs in response to PDGF-AA, and C3aR^{-/-}C5aR^{-/-} SMCs showed reduced proliferation. These findings show that C3aR/C5aR signal transduction in SMCs is important to their proliferative response in EC injury and atherogenesis. Results below show that a) atherogenesis is accelerated in the absence of DAF and b) the neointimal response to wire injury is markedly heightened in Daf1^{-/-} mice but suppressed below that in WTs in Daf1^{-/-}C3aR^{-/-} and Daf1^{-/-}C5aR^{-/-} mice are consistent with this interpretation.

Antagonizing C3aR/C5aR signal transduction induces the synthesis and activation of TGF-β1. We investigated whether TGF-β1 production might underlie the growth suppression connected with C3aR/C5aR blockade. To test this, 1) we assayed supernatants from NIH-3T3 cells treated with PDGF-AA+anti-C3a/anti-C5a mAbs and 2) following removal of anti-C3a/anti-C5a from the mAb treated NIH-3T3 cell cultures (with protein-G beads), we added supernatants without or with anti-TGF-β1 blocking antibody to fresh cultures of PDGF-AA treated NIH-3T3 cells. These analyses showed that antagonizing C3aR/C5aR signaling induced active TGF-β1 expression (+2.5 ng/ml) and that the elicited TGF-β1 suppressed PDGF-AA induced growth (5-fold). RT-PCR of PDGF-AA treated NIH-3T3 cells showed that PDGF-AA suppressed TGF-β1 mRNA transcription (˜3-fold), whereas C3aR/C5aR antagonism induced it. Activation of latent TGF-β1 can be dependent on Thrombospondin 1 (Tsp-1). Addition of anti-Tsp-1 Ab blocked the generation of active TGF-β1, indicating that Tsp-1 induction is also dependent on C3aR/C5aR antagonism (a result relevant to the pro-atherogenic phenotype of Tsp-1^{-/-} mice). Collectively, these findings indicate that 1) disruption of C3aR/C5aR signaling evokes TGF-β1 production, 2) TGF-β1 enters into an autocrine signaling loop, and 3) C3aR/C5aR signals and TGF-β1 signals oppose each other in regulating PDGF induced growth.

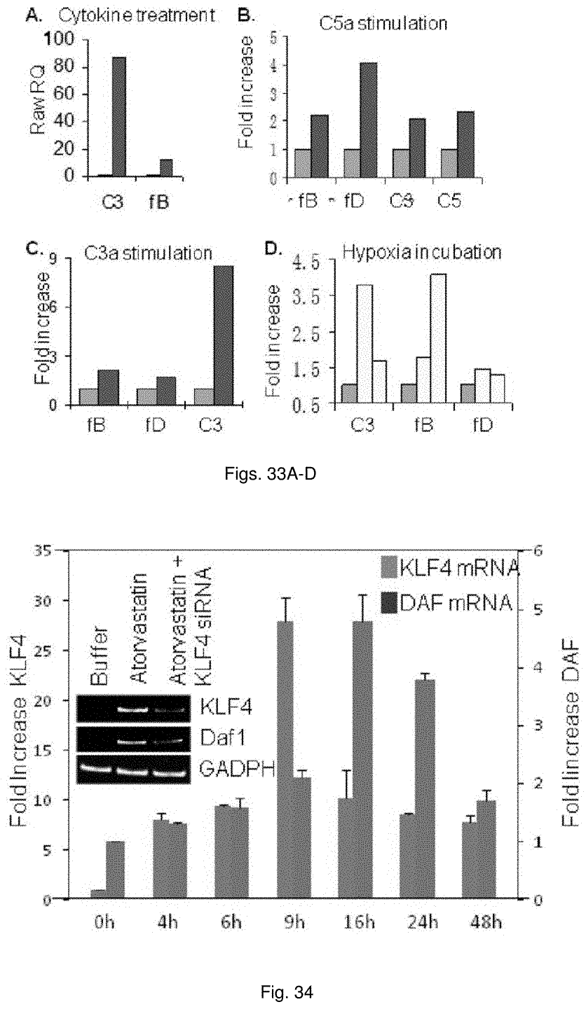

ECs and leukocytes locally synthesize C3/fB/fD/C5 and the elicited C3a/C5a signal through C3aR/C5aR on the same cells to amplify this synthesis. To determine whether ECs locally synthesize AP complement components, we added IL-1/IFN-γ/TNF-α to HUVEC, 1 hr after which we quantified C3 and fB transcripts by qPCR. This analysis (FIG. 33A) showed marked upregulation of both mRNAs. Consistent with the ability of C3a/C5a to feed back through C3aR/C5aR and initiate an auto-inductive signaling loop, added C3a or C5a caused the ECs to make more AP components (FIGS. 33B-C). Incubation of HUVEC with the mitochondrial uncoupler protonophore carbonyl cyanide p-(trifluoromethoxy)phenylhydrazone (FCCP)+iodoacetate (IAA) (which simulate hypoxia when added to cells) also increased HUVEC complement synthesis (FIG. 33D). To test whether leukocytes that are recruited to sites of EC injury locally synthesize AP components and C5a similarly feeds back in them to amplify local complement production, we stimulated Raji, Jurkat, U937 cells, primary mΦs, B cells, and DCs with 10 nM C5a. This documented induction of AP and C5 gene transcription by qPCR.

### DAF Attenuates Atherogenesis

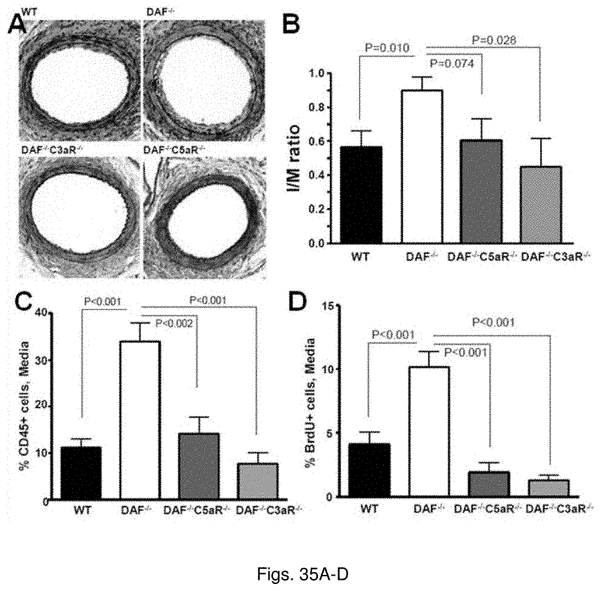

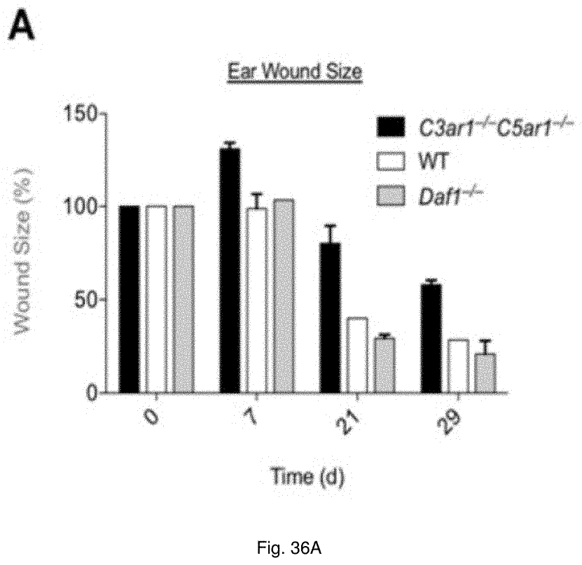

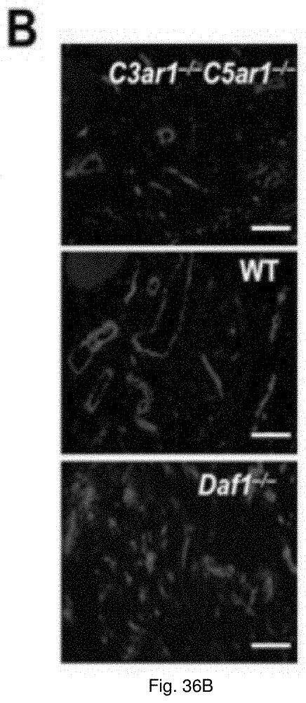

To determine whether DAF's inhibition of C3/C5 activation retards atherogenesis, we crossed Daf1^{-/-} mice with ApoE^{-/-} or ldlr^{-/-} mice, the two strains widely used to assess experimental atherosclerosis following high fat feeding. After placing homozygous Daf1^{-/-} ApoE^{-/-} and Daf1^{-/-}ldlr^{-/-} and their respective Daf1^{+/+} littermates on a high fat diet to accelerate atherogenesis, we initially compared their coronary arteries and aortas for plaques at wk 11. On both backgrounds, immunohistochemical analyses showed 1.5-2-fold larger plaques (μ2/section), Moreover, there was more C3b deposition in DAF's absence. In a second cohort, we quantified lipid accumulation by Oil Red-O staining after 17 weeks of a high fat diet. Both gross and histologic analyses (FIG. 34) revealed a robust increase in atherosclerotic burden in Daf1-null animals.

In view of the connections of MCP-1 and other proinflammatory cytokines with atherosclerosis and the mechanistic linkage of DAF deficiency with potentiated C3aR/C5aR transduction, we tested whether C5aR signaling induces proinflammatory gene transcription. Added C5a induced the mRNA transcription of chemotactic MCP-1/IL-8, proinflammatory IL-1β/IL-6 and growth inductive GM-CSF.

### DAF Regulates the Biological Response to EC Injury

To directly test whether DAF regulates inflammatory and proliferative responses to EC injury, we compared femoral artery wire injury in Daf1^{-/-} mice and WT controls. In this model, wire injury is accompanied by EC denudation, platelet and fibrin deposition, and prominent vascular inflammation. This model is used to closely mimic restenosis as seen following angioplasty in human patients. In WTs, we found that intimal thickening was evident 5 days post injury and further progressed between 5 and 14 days. Remarkably in Daf1^{-/-} mice, at day 14, intimal thickening was 155% greater (p=0.026) (FIG. 35A) and the intima:media area ratio (I:M) was ˜2-fold increased (0.90±0.20 vs. 0.57±0.41; p=0.01) compared to WT mice. Immunohistochemical staining for C3dg, a C3b fragment, was significantly increased in Daf1^{-/-} vs WT vessels (51.1±8.3% vs 30.6±10.0%; p=0.008; data not shown; FIG. 35B). In addition, Daf1^{-/-} vessels exhibited increased immune cell infiltration (CD45+ staining; FIG. 35C) and cellular proliferation (BrdU staining; FIG. 35D). These studies identify a direct role for DAF in the proliferative and inflammatory response to mechanical vascular injury. To establish whether the mechanism by which its deficiency alters the vascular response to EC injury is increased by C3aR and/or C5aR signaling, we repeated the studies in Daf1^{-/-}C5aR^{-/-} and Daf1^{-/-}C3aR^{-/-} mice. We found that the augmented vascular inflammation, cellular proliferation, and neointimal thickening observed in Daf1^{-/-} mice decreased to, or even below, WT levels in each KO animal (FIG. 35). In summary, the results demonstrated that DAF control of C3a/C5a generation and consequent C3aR/C5aR signaling is critical for the biological response to EC injury. These finding were recently confirmed in studies employing C3aR/C5aR antagonists.

### Example 3

We hypothesized that DAF deficiency or blockade might accelerate wound healing, whereas C3ar1/C5ar1 deficiency might retard it. For testing this, we exploited Daf1^{-/-}, WT, and C3ar1^{-/-}C5ar1^{-/-} mice (each congenic on C57BL/6) and compared the rates of wound healing in four independent wound models. With the use of a mouse anti-mouse DAF Ab directed against DAF's active site, we then went on to test whether blockade of DAF in WT mice would accelerate wound healing.

### Methods

### Mice and Reagents

WT C56BL/6 mice were purchased from Jackson Labs. Daf1^{-/-} and C3ar1^{-/-}C5ar1^{-/-} were bred 12 generations on the C57BL/6 background as described previously. Mice aged 7-12 wk were used for all models. Mice were housed in a specific pathogen-free facility and were confirmed to be negative for common murine viral pathogens by routine sera analysis. Experiments were conducted by following established guidelines for animal care, and all protocols were approved by the appropriate institutional committees.

### Ear Wound Model

Female mice of each genotype were anesthetized. The ear was punctured using a 5 mm biopsy punch. The size of the wound was measured on days (d) 7, 21, and 29 after the procedure.

### Syngeneic Skin Transplant Model

Adult recipient mice were shaved and depilated 3 d prior to transplantation. Mice were anesthetized. For skin experiments, a 2×2-cm midline area of their backs were delineated, shaved and depilated. The dorsal area was prepared for wounding or denervation using a Betadine scrub and wiping with 70% alcohol. All instruments used in any of the procedures were either sterile disposable or sterilized prior to use. The skin (˜1.0-1.5 cm²) transplant from the donor mouse (cut to the same size) was secured in place using either sutures or Band-Aid, the graft was protected from injury by bandaging the area following transplant. Clinical observations and assessments were made daily throughout the experiment and was recorded. Photographs of the grafts were taken at least twice for all animals. Mice were monitored daily and mice showing any signs of morbidity (site infection, poor healing (redness and itching disappearance in 1-3 d is accepted as a sign of healing) mice losing more than 15% of their body mass, and/or the animals were non-ambulatory, etc.) were euthanized. All skin samples were divided, and half processed for histology, and half used for CD31 analyses. Tissue to be used for histology/ISH was fixed in 10% neutral buffered formalin for 16 hours, then transferred to 70% ethyl alcohol and paraffin-embedded. The fixation time duration was recorded for each sample. Tissues to be used for RT-PCR were frozen in liquid nitrogen and stored at −80° C.

### Burn Model

The backs of 8-10 week-old mice were shaved and depilated one day prior to the experiment and given normal food and access to water. Immediately prior to the burn injury, mice were anesthetized with ketamine (8-10 mg/100 g), xylazine (0.5-1.0 mg/100 g), and acepromazine (0.3 mg/100 g) intraperitoneally (i.p.) and the exposed skin cleaned with 70% ethanol. A consistent full-thickness burn wound was performed by heating an insulated 8 mm diameter steel rod to 120° C. and placing the rod on the shaved area for 20 seconds (s). No additional pressure was applied to the rod besides the weight of the rod itself. Buprenorphine (0.3 mg/ml) and 0.9% saline were given i.p. for analgesia and fluid resuscitation immediately after injury. The animals were observed daily and sacrificed if signs of distress were apparent. Wound areas were photographed at 0, 3, 6, and 9 d following the procedure and the change in size of the wound area was analyzed using MetaMorph. Mice were sacrificed 14 d after the injury by CO₂ asphyxiation. Burn wound skins and surrounding area were harvested and fixed in 4% paraformaldehyde for 24 hours (h) and stained with H&E.

### Corneal Epithelialization

Mice anesthetized with isoflurane chamber were placed in an induction chamber connected to an oxygen source and isoflurane vaporizer adjusting oxygen flow and to 0.9 liters/min and the isoflurane vaporizer to 1-2%. As soon as the mouse became unresponsive and had shallow breathing, it was transferred to the anesthesia platform with the nose cone in place and placed on a heating pad. One drop of 1% proparacaine hydrochloride was applied to the right eye prior to the procedure. After marking the cornea with a 1.5 mm trephine, a uniform circular 5-6 cell epithelial layer was removed with an Algerbrush II with 0.5 mm Burr (The Alger company, Inc, TX, USA). Fluorescein solution was applied to stain the cornea to measure the size of the defect.

### DAF Blockade on Corneal Wound Healing

For studies of DAF blockade, WT mice were treated with a 20 μL drop of mouse anti-mouse DAF antibody at 1:50 diluted plasma. At same time intervals, another group of WT mice were treated with pre-immunization plasma. Procedures were performed under a Leica stereo microscope with a fixed magnification and brightness in order to maintain consistency. Pictures were taken using the green fluorescent protein filter of the microscope at 0, 4, 12 and 24 h following the procedure and the size decrease of the de-epithelialized area was analyzed using MetaMorph. The other groups were not treated and photographed at their natural course.

### Preparation of Mouse Anti-Mouse Daf1 CCP2-3 Antibodies

Twelve C57BL/6 Daf1^{-/-} were immunized with recombinant mouse DAF CCPs2-3 (60 μg/mouse) emulsified with complete Freund's adjuvant (CFA) or with PBS as control. Blood was collected from the tail vein of each mouse prior to immunization and weekly thereafter. Plasmas were applied to an ELISA to assess levels of anti-Daf1CCPs2-3 antibodies.

### Immunohistochemistry

Frozen skin sections of the graft bed were allowed to air dry for 30 minutes at room temperature. They were fixed in cold acetone for 10 minutes and rinsed with PBS. The sections were blocked with 4% normal bovine serum for 1 hour at room temperature. Rat anti-mouse CD31 (15.6 μg/ml) from BD Biosciences at 1:100 was applied and incubated at 4° C. overnight followed by washings with PBS for three times for 10 minutes each. The sections were then incubated with Alexa Fluor 594 goat anti-rat IgG2a (2 mg/ml) for one hour. ProLong Gold Antifade Mounting Media (Life Technologies) was applied on the sections and coverslipped.

### Statistics

Statistical significance for all experimental data was determined by Student's t-test (unpaired, two-tailed) with Microsoft Excel or GraphPad Prism 5.

### Results

### Ear Wound Healing

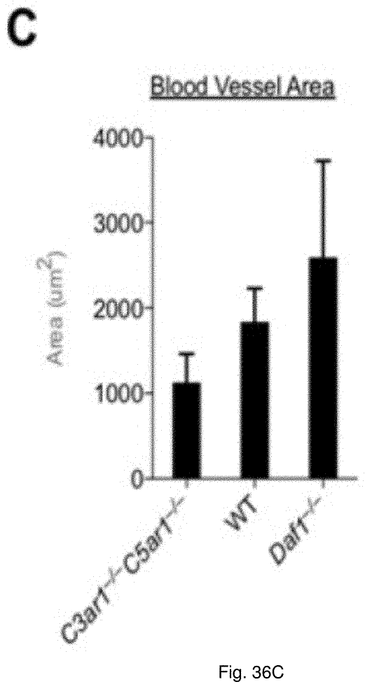

In initial studies, we examined wound closure following ear lobe puncture, a model widely used by others. We made equivalent 5 mm punctures in the ear lobes of WT, Daf1^{-/-} and C3ar1^{-/-}C5ar1^{-/-} mice (3 mice in each group) and measured the area of the puncture opening daily. At d 29 post puncture, the 5-mm wound declined slowly reaching 44.2% of the original size in C3ar1^{-/-}C5ar1^{-/-} mice, whereas it reached 13.9% of the original size in Daf1^{-/-} mice, a >200% more rapid decrease compared to C3ar1^{-/-}C5ar1^{-/-} mice. This compared to 31.1% in WT mice (FIG. 36A).

### Skin Transplant Engraftment

We next studied skin engraftment using re-grafted autologous skin to eliminate adaptive immune responses. Two by 2 cm skin grafts excised from the left flanks of each of the three genotypes (4 mice each group) were immediately re implanted in the same site and covered with gauze. Analysis of the grafts 14 d post-engraftment showed that the transplanted skin in Daf1^{-/-} mice re-granulated ˜90% faster than in that in WTs. Conversely, the transplanted skin in C3ar1^{-/-}C5ar1^{-/-} mice showed ˜70% delayed re-granulation with less hair regrowth. Quantitation of blood vessels at the junction of the transplant showed 50% higher blood vessel density in Daf1^{-/-} mice than in WTs as opposed to ˜400% lower density in C3ar1^{-/-}C5ar1^{-/-} mice (FIGS. 36B, C).

### Burn Recovery

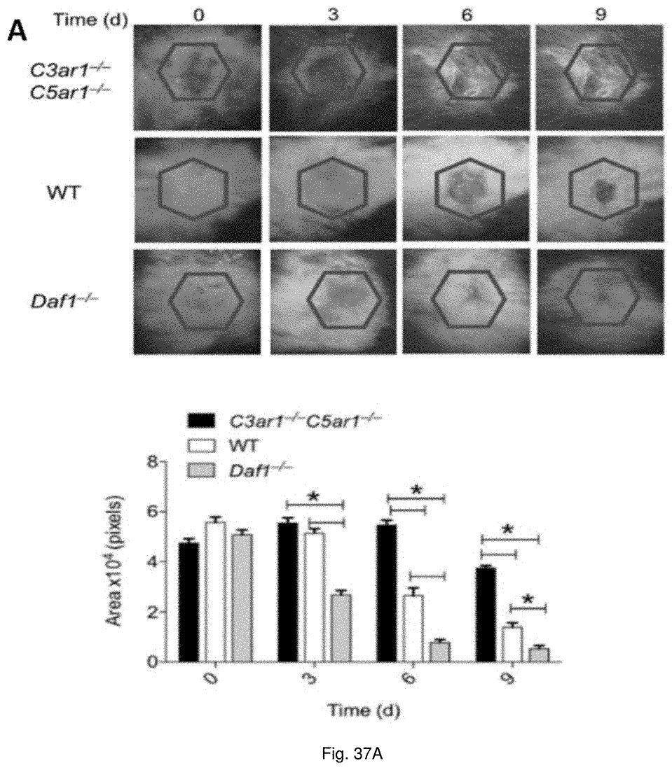

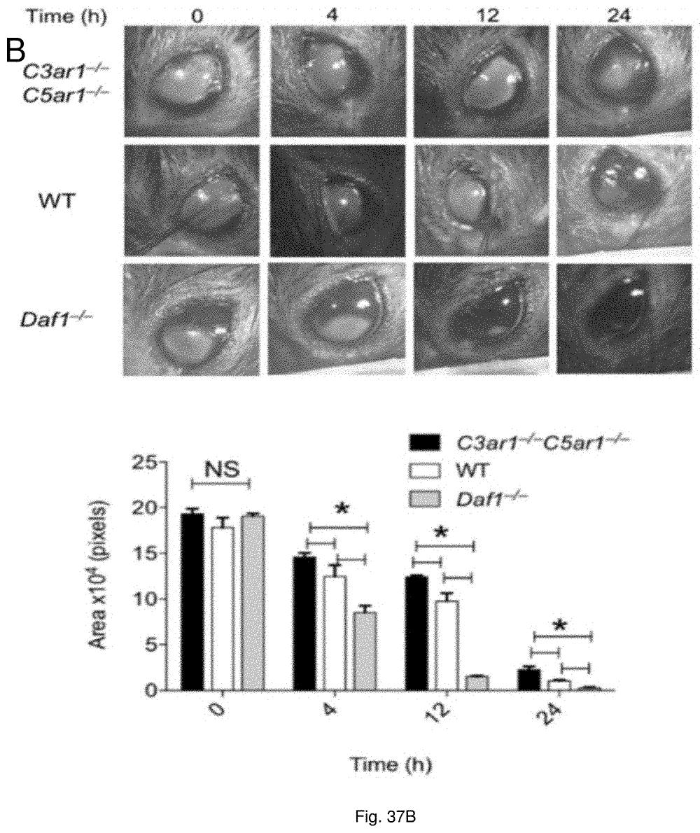

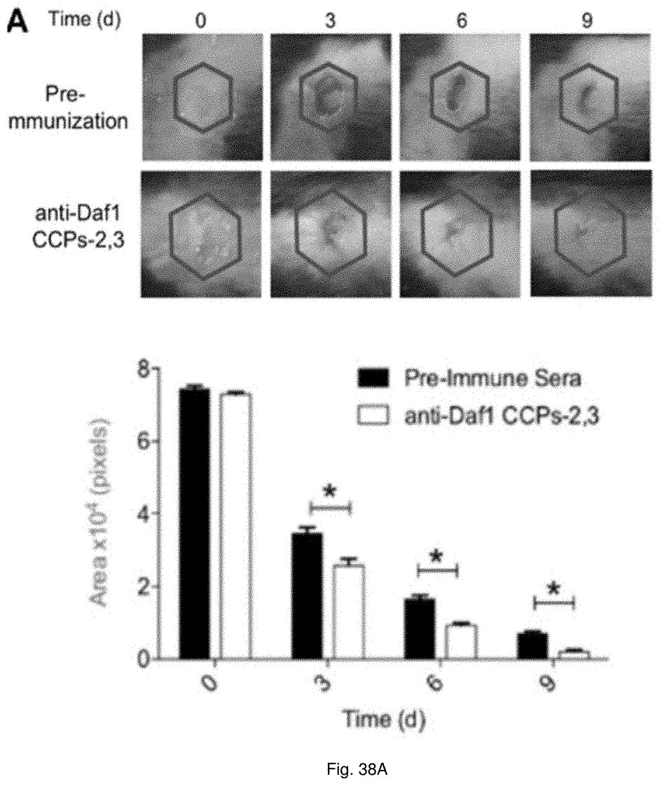

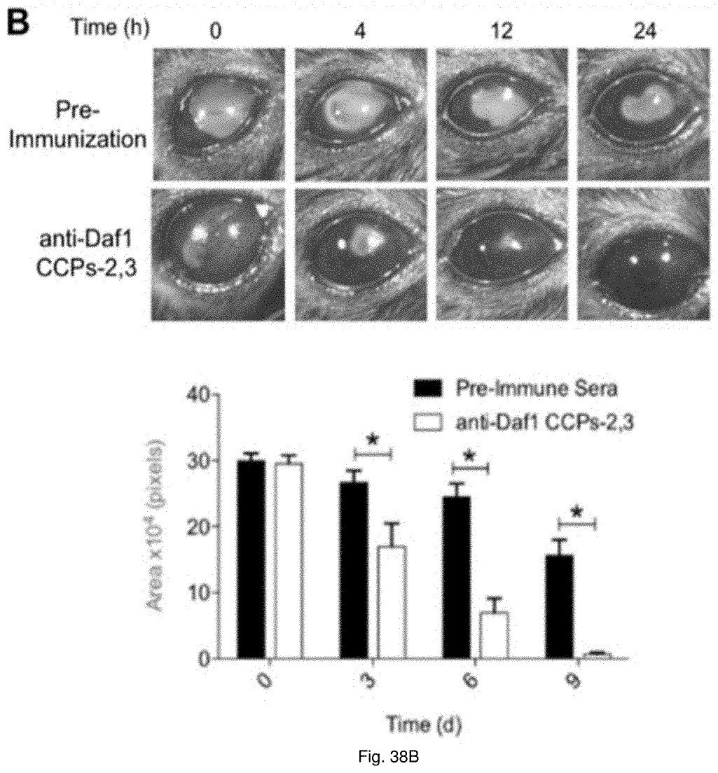

In a third model, we examined recovery from burn injury. Round 0.8 cm burns were made by placing a heated (120°) metal rod on shaved skin on the back of the mice in each genotype. All mice survived and displayed no sign of prolonged pain. The burn wound diameter decreased ˜150% faster in Daf1^{-/-} mice (n=3) compared to WT mice (n=3) (FIG. 37A). This contrasted with >200% lesser decrease in burn diameter in C3ar1^{-/-}C5ar1^{-/-} mice (n=11).

### Re-Epithelialization of the Corneas

In a fourth model, we compared healing of injured corneas. A uniform 1.5 mm circular 5-6 cell epithelial layer of corneal epithelium was removed after marking the site with a 1.5 mm trephine employing an Algerbrush II with a 0.5 mm Burr. After recovery from anesthesia, corneas were stained with fluorescein to quantitate the wound size. Wound size was measured at time 0, and 4, 8, and 24 h after injury by re-staining with fluorescein. The corneas of Daf1^{-/-} mice re-epithelialized 200% faster than those of WTs, whereas those of C3ar1^{-/-}C5ar1^{-/-} mice re-epithelized 150% slower that WTs (FIG. 37B).

### Acceleration of Wound Healing by Blockade of DAF

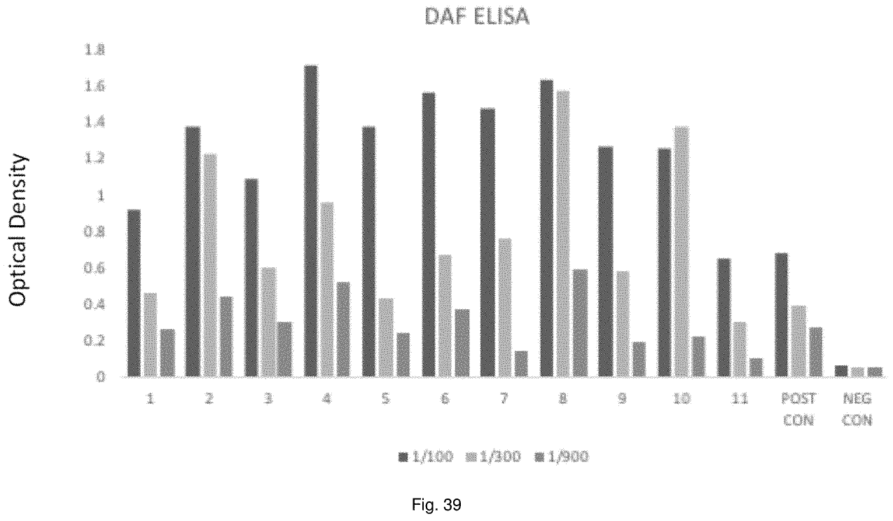

In all four models, deficiency of DAF [which potentiates autocrine C3ar1/C5ar1 signaling] markedly accelerated wound healing. Conversely, deficiency of C3ar1/C5ar1 [which disables autocrine C3ar1/C5ar1 signaling] uniformly slowed healing. These comparable differences between the genotypes in all 4 models prompted the question of whether DAF blockade in WT mice would similarly accelerate wound healing, a procedure which could be exploited clinically. To test this, we developed a mouse anti-DAF Ab against DAF's complement control protein repeats (CCPs23) which contain its active site. We accomplished the specificity by immunizing our Daf1^{-/-} mice selectively devoid of CCP23 with full length recombinant mouse Daf1 protein. This yielded Abs selective for CCPs23 of DAF because the other regions of the full length DAF immunogen are homologous with the recipient DAF. ELISAs of plasmas from 11 different immunized Daf1^{-/-} mice identified the anti-DAF CCPs23 Ab possessing the highest anti-DAF activity FIG. 39. We then used that anti-DAF plasma together with pre-immunization control plasma in WT mice subjected to burn wound or to corneal injury to test whether DAF blockade would have the same effect as DAF deficiency in accelerating healing.

To test whether the anti-Daf1 CCP23 Ab would accelerate healing in the burn model, we covered burn wounds on WT mice with bandages (3M) presoaked in mouse anti-mouse Daf1 CCP23 plasma or with pre-immunization plasma (6 mice each group). An additional 6 mice received bandages presoaked in PBS as a control. Comparison of wound sizes daily showed that bandages presoaked in the anti-Daf1 CCP23 plasma accelerated burn repair 150-250% faster than those presoaked in nonimmune plasma or PBS (FIG. 38A).

To determine if the DAF blockade with the anti-CCP23 Ab would similarly promote corneal wound healing, we produced circular epithelial layer lesions in 12 WT mice as done above. We added 20 μl of 1:50 diluted anti-Daf1 plasma at time 0, 2 and 6 h to the tears of 6 mice and added the same amount of pre-immune plasma to the tears of 6 identically injured mice. We then stained eyes with fluorescein at 0, 6, 12, and 24 h as in the above studies comparing the three genotypes. The corneas of the mice treated with anti-mouse Daf1 CCP23 plasma healed 350% faster than the corneas of mice treated with pre-immune plasma (FIG. 38B). Side by side comparisons of the kinetics of healing (FIG. 3 panels A vs B) showed the rate of healing approximated that in Daf1^{-/-} mice. Taken together with the burn model, the data argued that the anti-DAF CCPs23 blockade could be effective to accelerate wound healing therapeutically.

During the course of examining the effect of DAF blockade in the two injury models using anti-DAF CCPs23 Ab containing plasma, we prepared anti-mouse DAFCCPs23 mAbs (FIG. 39). The mAbs will enable studies of the efficacy of DAF blockade in more extensive injury models, e.g. severe generalized burns, fractures, or other major traumas.

From the above description of the invention, those skilled in the art will perceive improvements, changes and modifications Such improvements, changes and modifications are within the skill of the art and are intended to be covered by the appended claims. All publications, patents, and patent applications cited in the present application are herein incorporated by reference in their entirety.

Having described the invention, we claim:

1. A method of promoting wound healing in a subject in need thereof, the method comprising:
administering to cells proximate or about the periphery of a wound of the subject at least one agent that enhances C3aR and/or C5aR signaling of the cells.

2. The method of claim 1, wherein the agent substantially increases the interaction of at least one of C3a or C5a with the C3a receptor (C3aR) and C5a receptor (C5aR).

3. The method of claim 2, wherein the agent comprises at least one of DAF antagonist, C3aR agonist and/or a C5aR agonist.

4. The method of claim 3, wherein the DAF antagonist comprises an anti-DAF antibody or antigen binding fragment thereof that binds to DAF and to reduce DAF inhibition and/or degradation of C3 convertase and/or C5 convertase.

5. The method of claim 4, wherein the anti-DAF antibody or antigen binding fragment thereof binds to CCP2 and/or CCP3 of DAF.

6. The method of claim 1, wherein the agent comprising DAF interfering RNA.

7. The method of claim 1, wherein the agent comprises a C3aR agonist and/or a C5aR agonist that enhances C3aR and/or C5aR signaling of the cells.

8. The method of claim 1, the agent being administered to the wound of the subject at an amount effective to promote at least one of growth, viability, or mitosis of cells proximate or about the periphery of the wound.

9. The method of claim 1, wherein the wounds include acute injuries or wounds, damage to bodily tissues, injuries sustained during medical procedures, trauma-induced injuries, ulcers, post-surgical injuries, chronic wounds, acne, dermatitis, wounds following dental surgery, periodontal disease, and tumor associated wounds.

10. The method of claim 9, wherein the wounds comprise at least one of such as thermal burns, chemical burns, radiation burns, burns caused by excess exposure to ultraviolet radiation, damage to bodily tissues as a result of labor and childbirth, cuts, incisions, excoriations, injuries sustained as result of accidents, pressure ulcers, diabetic ulcers, plaster ulcers, decubitus ulcer, post-surgical injuries, pressure sores, bedsores, acne, impetigo, intertrigo, folliculitis, or eczema.

11. A method of treating a wound in a subject in need thereof, the method comprising:
administering to cells proximate or about the periphery of the wound at least one agent that enhances C3aR and/or C5aR signaling of the cells.

12. The method of claim 11, wherein the agent substantially increases the interaction of at least one of C3a or C5a with the C3a receptor (C3aR) and C5a receptor (C5aR).

13. The method of claim 12, wherein the agent comprises at least one of DAF antagonist, C3aR agonist and/or a C5aR agonist.

14. The method of claim 13, wherein the DAF antagonist comprises an anti-DAF antibody or antigen binding fragment thereof that binds to DAF and to reduce DAF inhibition and/or degradation of C3 convertase and/or C5 convertase.

15. The method of claim 14, wherein the anti-DAF antibody or antigen binding fragment thereof binds to CCP2 and/or CCP3 of DAF.

16. The method of claim 11, wherein the agent comprising DAF interfering RNA.

17. The method of claim 11, wherein the agent comprises a C3aR agonist and/or a C5aR agonist that enhances C3aR and/or C5aR signaling of the cells.

18. The method of claim 11, the agent being administered to the wound of the subject at an amount effective to promote at least one of growth, viability, or mitosis of cells proximate or about the periphery of the wound.

19. The method of claim 11, wherein the wounds include acute injuries or wounds, damage to bodily tissues, injuries sustained during medical procedures, trauma-induced injuries, ulcers, post-surgical injuries, chronic wounds, acne, dermatitis, wounds following dental surgery, periodontal disease, and tumor associated wounds.

20. The method of claim 19, wherein the wounds comprise at least one of such as thermal burns, chemical burns, radiation burns, burns caused by excess exposure to ultraviolet radiation, damage to bodily tissues as a result of labor and childbirth, cuts, incisions, excoriations, injuries sustained as result of accidents, pressure ulcers, diabetic ulcers, plaster ulcers, decubitus ulcer, post-surgical injuries, pressure sores, bedsores, acne, impetigo, intertrigo, folliculitis, or eczema.


SEQUENCE LISTING (4 sequences)

SEQ ID NO: 1 - DNA, 19 bp, Artificial Sequence
  Synthetic Construct
gaagaguucugcaaucgua

SEQ ID NO: 2 - DNA, 19 bp, Artificial Sequence
  Synthetic Construct
uacgauugcagaacucuuc

SEQ ID NO: 3 - protein, 15 aa, Artificial Sequence
  Synthetic Construct
WWGKKYRASKLGLAR

SEQ ID NO: 4 - protein, 10 aa, Artificial Sequence
  Synthetic Construct
YSFKPMPLAR